(19) United States
(12) Patent Application Publication
(10) Pub. No.: US 2009/0246752 A1
(43) Pub. Date: Oct. 1, 2009

(54) APPARATUS AND METHOD FOR DETECTING MICROSCOPIC LIVING ORGANISMS USING BACTERIOPHAGE

(75) Inventors: Kent Voorhees, Golden, CO (US); Jon Rees, Longmont, CO (US); John H. Wheeler, Boulder, CO (US); Angelo Madonna, Tooele, UT (US)

Correspondence Address: PATTON BOGGS LLP, 1801 CALFORNIA STREET, SUITE 4900, DENVER, CO, 80202, US

(73) Assignee: MicroPhage TM Incorporated, Longmont, CO (US)

(21) Appl. No.: 12/346,656

(22) Filed: Dec. 30, 2008

Related U.S. Application Data
(62) Division of application No. 10/823,294, filed on Apr. 12, 2004, now abandoned.
Application No. 10/823,294 is a continuation-in-part of application No. 10/249,452, filed on Apr. 10, 2003, now Pat. No. 7,166,425.
(60) Provisional application No. 60/319,184, filed on Apr. 12, 2002.
(60) Provisional application No. 60/544,437, filed on Feb. 13, 2004.
(60) Provisional application No. 60/557,962, filed on Mar. 31, 2004.

(51) Int. Cl. C12Q 1/70

(52) U.S. Cl. 435/5

(57) ABSTRACT

A method for detecting one or more target bacteria in a raw sample where: 1) bacteriophage(s) specific to each target bacterium are added to the raw sample, 2) the test sample is incubated, and 3) the test sample is tested for the presence of each phage in sufficient numbers to indicate the presence of the associated target bacteria in the raw sample. In one embodiment, each phage is initially added to the raw sample in concentrations below the detection limit of the final phage detection process. In another embodiment, the parent phages are tagged in such a way that they can be separated from the progeny phage prior to the detection process. Preferred phage detection processes are immunoassay methods utilizing antibodies that bind specifically to each phage. Antibodies can be used that bind to the protein capsid of the phage. Alternatively, the phage can by dissociated after the incubation process and the sample tested for the presence of individual capsid proteins or phage nucleic acids. The invention can be used to test target bacteria for antibiotic resistance.

### CROSS-REFERENCE TO RELATED APPLICATIONS

This application is a divisional of U.S. patent application Ser. No. 10/823,294 filed Apr. 12, 2004, which is a continuation-in-part of U.S. patent application Ser. No. 10/249,452 filed Apr. 10, 2003, now U.S. Pat. No. 7,166,425 issued Jan. 23, 2007, which claims the benefit of U.S. Provisional Application No. 60/319,184 filed Apr. 12, 2002. U.S. patent application Ser. No. 10/823,294 also claims the benefit of U.S. Provisional Application No. 60/544,437 filed Feb. 13, 2004 and U.S. Provisional Application No. 60/557,962 filed Mar. 31, 2004. All of the above patent applications, both provisional and non-provisional, are hereby incorporated by reference to the same extent as though fully contained herein.

### FIELD OF THE INVENTION

The invention relates generally to the field of detection of microscopic living organisms, and more particularly to the detection of bacteria utilizing bacteriophage.

### BACKGROUND OF THE INVENTION

Standard microbiological methods for detection of microorganisms have relied on substrate-based assays to test for the presence of specific bacterial pathogens. See Robert H. Bordner, John A. Winter, and Pasquale Scarpino, Microbological Methods For Monitoring The Environment, EPA Report No. EPA-600/8-78-017, U.S. Environmental Protection Agency, Cincinnati, Ohio, 45268, December 1978. These techniques are generally easy to perform, do not require expensive supplies or laboratory facilities, and offer high levels of selectivity. However, these methods are slow. Substrate-based assays are hindered by the requirement to first grow or cultivate pure cultures of the targeted organism, which can take twenty-four hours or longer. This time constraint severely limits the effectiveness to provide rapid response to the presence of virulent strains of microorganisms.

Molecular biology techniques are quickly gaining acceptance as valuable alternatives to standard microbiological tests. Serological methods have been widely employed to evaluate a host of matrices for targeted microorganisms. See David T. Kingsbury and Stanley Falkow, Rapid Detection And Identification of Infectious Agents, Academic Press, Inc., New York, 1985 and G. M. Wyatt, H. A. Lee, and M. R. A. Morgan, Chapman & Hall, New York, 1992. These tests focus on using antibodies to first trap and then separate targeted organisms from other constituents in complicated biological mixtures. Once isolated, the captured organism can be concentrated and detected by a variety of different techniques that do not require cultivating the biological analyte. One such approach, termed “immunomagnetic separation” (IMS), involves immobilizing antibodies to spherical, micro-sized magnetic or paramagnetic beads and using these beads to trap targeted microorganisms from liquid media. The beads are easily manipulated under the influence of a magnetic field facilitating the retrieval and concentration of targeted organisms. Moreover, the small size and shape of the beads allow them to become evenly dispersed in the sample, accelerating the rate of interaction between bead and target. These favorable characteristics lead to reductions in assay time and help streamline the analytical procedure, making it more applicable for higher sample throughput and automation.

Downstream detection methods previously used with IMS include ELISA (Kofitsyo S. Cudjoe, Therese Hagtvedt, and Richard Dainty, “Immunomagnetic Separation of Salmonella From Foods And Their Detection Using Immunomagnetic Particle”, International Journal of Food Microbiology, 27 (1995), pp. 11-25), dot blot assay (Eystein Skjerve, Liv Marit Rorvik, and Orjan Olsvick, “Detection Of Listeria Monocytogenes In Foods By Immunomagnetic Separation”, Applied and Environmental Microbiology, November 1990, pp. 3478-3481), electrochemiluminescence (Hao Yu and John G. Bruno, Immunomagnetic-Electrochemiluminescent Detection Of Escherichia coli O157 and Salmonella typhimurium In Foods and Environmental Water Samples”, Applied and Environmental Microbiology, February 1996, pp. 587-592), and flow cytometry (Barry H. Pyle, Susan C. Broadway, and Gordon A. McFeters, “Sensitive Detection of Escherichia coli O157:H7 In Food and Water By Immunomagnetic Separation And Solid-Phase Laser Cytometry”, Applied and Environmental Microbiology, May 1999, pp. 1966-1972). Although these tests provide satisfactory results, they are laborious to perform and give binary responses (yes/no) that are highly susceptible to false-positive results due to cross-reactivity with non-target analytes. Another method for identifying whole cellular microorganisms uses IMS coupled to matrix-assisted laser desorption/ionization (MALDI) time-of-flight (TOF) mass spectrometry (MS) (Holland et al., 1996; van Barr, 2000; Madonna et al., 2000).

All of these newer approaches can offer faster results than do traditional microbiology methods. However, they do not achieve the sensitivity levels that substrate-based assays do, are more expensive, and typically require more highly trained technicians than do classical substrate-based methods.

Other molecular biology techniques that have received a great deal of attention recently are Polymerase Chain Reaction (PCR) methods. PCR detection of specific microorganisms in a sample involves extraction of the genetic material (RNA and/or DNA) in a sample, amplification of a target genetic sequence specific to the microorganism of interest, and then detection of the amplified genetic material. PCR techniques offer high selectivity owing to the uniqueness of the detected genetic material, high sensitivity because of the substantial amplification of the target genetic material, and rapid results owing to the potentially fast amplification process. However, PCR instruments and reagents are quite expensive and highly trained technicians are needed to perform the tests. To date, PCR instruments have not delivered the hoped-for sensitivity or specificity.

Some attempts have been made to improve upon substrate-based classical bacterial detection methods using bacteriophage infection and/or amplification. Bacteriophages are viruses that have evolved in nature to use bacteria as a means of replicating themselves. A bacteriophage (or phage) does this by attaching itself to a bacterium and injecting its genetic material into that bacterium, inducing it to replicate the phage from tens to thousands of times. Some bacteriophage, called lytic bacteriophage, rupture the host bacterium releasing the progeny phage into the environment to seek out other bacteria. The total incubation time for phage infection of a bacterium, phage multiplication (amplification) in the bacterium, and release of the progeny phage after lysis can take as little as an hour depending on the phage, the bacterium, and the environmental conditions. Microbiologists have isolated and characterized over 5,000 phage species, including many that specifically target bacteria at the species or even the strain level. U.S. Pat. No. 5,985,596 issued Nov. 16, 1999 to Stuart Mark Wilson and U.S. Pat. No. 6,461,833 B1 issued Oct. 8, 2002 to Stuart Mark Wilson describe such a phage-based assay method. It comprises a lytic phage infection of a sample that may contain bacteria of interest. This is followed by removal of free phage from the sample, target bacteria lysis, and then infection of a second bacterium by the progeny phage where the second bacterium has a shorter doubling time than does the target bacterium. The prepared sample is grown on a substrate and the formation of plaques indicates the presence of the target bacterium in the original sample. This method can shorten the assay time of a traditional substrate-based assay, though assays still take many hours or days because of the requisite culture incubation times. Another problem with the method is that it can only be applied to detect bacterium for which a non-specific phage exists that also infects a more rapidly doubling bacterium than the target bacterium. Usage of a nonspecific phage opens the possibility of cross-reactivity to at least the second bacterium in test samples. Thus, this phage-based, plaque assay method is not rapid, can only be applied if a suitable non-specific phage is available, is prone to cross-reactivity problems, and must be performed in a lab setting.

Other bacterial pathogen detection methods have abandoned the substrate-based, plaque detection methodology altogether. Many of these methods utilize bacteriophage that have been genetically modified with a lux gene which is only expressed if a target bacterium is present in a sample and is then infected by the modified phage. U.S. Pat. No. 4,861,709 issued Aug. 29, 1989 to Ulitzur et al. is a typical example. A phage that specifically infects a target pathogen is modified to include a lux gene. When the modified phage is added to a sample containing the target bacterium, the phage infects the bacterium, luciferase is produced in the bacterium, and light is emitted. U.S. Pat. No. 5,824,468 issued Oct. 20, 1998 to Scherer et al. describes a similar method. In addition to luciferase-producing gene markers, Scherer et al. describes gene markers that are expressed as detectable proteins or nucleic acids. U.S. Pat. No. 5,656,424 issued Aug. 12, 1997 to Jurgensen et al. describes a method utilizing luciferase (or β-galactosidase) reporter phage to detect mycobacteria. It further describes testing for antibiotic susceptibility. U.S. Pat. No. 6,300,061 B1 issued Oct. 9, 2001 to Jacobs, Jr. et al. describes yet another method for detecting mycobacteria using genetically modified phage, which produces one of several reporter molecules after bacterial infection, including luciferase. U.S. Pat. No. 6,555,312 B1 issued Apr. 29, 2003 to Hiroshi Nakayama describes a method utilizing a gene that produces a fluorescent protein marker rather than a luminescent one. All of these methods take implicit advantage of phage amplification within infected bacteria. For each target bacterium infected in a sample, the marker gene is expressed many times over as the progeny phage are produced. U.S. Pat. No. 6,544,729 B2 issued Apr. 8, 2003 to Sayler et al. adds an additional amplification process. A phage's DNA is modified to include a lux gene. A bioreporter cell is also modified to include a lux gene. The genetically modified phage and bioreporter cells are added to a sample. If the phage infects target bacteria, the target bacteria are induced to produce not only luciferase but also acyl en homoserine lactone N-(3-oxohexanoyl) homoserine lactone (AHL). AHL finds its way into the bioreporter cells, stimulating the production of additional light and additional AHL, which in turn finds its way into additional bioreporter cells resulting in the production of even more light. Thus, an amplified light signal is triggered by the phage infection of the target bacteria. In principle, all of these methods utilizing genetically modified phage make possible: 1) high selectivity because they utilize selectively infecting phage; 2) high sensitivity because the marker gene products can be detected at low levels; and 3) results that are faster than substrate-based methods because the signal can be detected within one or two phage infection cycles. They have two significant drawbacks. First, they are expensive and difficult to implement because suitable phage must be genetically modified for each pathogen to be tested. Second, they often require an instrument to detect the marker signal (light), driving up the cost of tests utilizing genetically modified phage.

U.S. Pat. No. 5,888,725 issued Mar. 30, 1999 to Michael F. Sanders describes a method utilizing unmodified, highly specific lytic phages to infect target bacteria in a sample. Phage-induced lysis releases certain nucleotides from the bacterial cell such as ATP that can be detected using known techniques. Detecting increased nucleotide concentrations in a sample after phage infection indicates the presence of target bacteria in the sample. U.S. Pat. No. 6,436,661 B1 issued Aug. 20, 2002 to Adams et al. describes a method whereby a phage is used to infect and lyse a target bacterium in a sample releasing intracellular enzymes, which react in turn with an immobilized enzyme substrate, thereby producing a detectable signal. While these methods have the advantage of using unmodified phage, they do not derive any benefit from phage amplification. The concentration of detected markers (nucleotides or enzymes) is directly proportional to the concentration of target bacteria in the sample.

U.S. Pat. No. 5,498,525 issued Mar. 12, 1996 to Rees et al. describes a pathogen detection method using unmodified phage and phage amplification to boost the detectable signal. The method calls for adding a high concentration of a lytic phage to a sample. The sample is incubated long enough to allow the phage to infect the target bacteria in the sample. Before lysis occurs, the sample is treated to remove, destroy, or otherwise inactivate the free phage in the sample without affecting the progeny phage being replicated within infected bacteria. If necessary, the sample is subsequently treated to neutralize the effects of any anti-viral agent previously added to the sample. The progeny phage released by lysis are detected using a direct assay of the progeny phage or by using a genetically modified bioreporter bacterium to generate a signal indicating the presence of progeny phage in the sample. In either case, the measured signal is proportional to the number of progeny phage rather than the number of target bacteria in the original sample and, thus, is enhanced as a result of phage amplification. A key disadvantage of this method is that it requires free phage in the treated sample to be destroyed, removed, or inactivated followed by reversal of the virucidal conditions such that progeny phage will remain viable after lysis. These additional processes complicate assays utilizing the method and make them more expensive.

What is needed is a detection method combining the sensitivity, simplicity, and/or low cost of substrate-based assays with the rapid results offered by molecular biology diagnostic tests.

### BRIEF SUMMARY OF THE INVENTION

The invention solves the above problems, as well as other problems of the prior art, by providing methods and apparatus for detecting living microorganisms using the principle of phage amplification. In the preferred embodiment, a phage that is specific to a target microorganism is introduced into a sample to be tested. The amount of phage that is introduced is preferably an amount below the detection limit of the phage. If the target microorganism is present, the phage infects and multiplies within the microorganism. Preferably, the microorganism is lysed, either naturally as a result of the phage multiplication, or by an active lysing process, such as, if the microorganism is a bacterium, a bacterial lysozyme. In one embodiment, the phage is dissociated, preferably by adding a bacteriophage dissociating agent. In another embodiment, the parent phage are tagged such that they can be physically removed or segregated from the progeny phage prior to the detection process, thereby increasing potential sensitivity and/or reducing the total analysis time of the method. The sample is then assayed for the phage or a biological substance associated with the bacteriophage. If any phage or the biological substance are detected, the presence of the targeted microorganism is indicated. If no phage or biological substance is detected, the absence of the targeted microorganism is indicated. The total incubation process consisting of infection, replication, and lysis can take only minutes. All of the foregoing embodiments can be used to determine the antibiotic resistance or susceptibility of bacteria and other microorganisms. The bacteriophage or biological substance can be detected in any suitable fashion, such as with a lateral flow strip, a SILAS surface, or by a MALDI mass spectrometer.

In one preferred embodiment, the invention provides a method of detecting the presence or absence of a microorganism in a sample to be tested, the method comprising: combining with the sample, parent bacteriophage capable of infecting the target microorganism to create a bacteriophage exposed sample; providing conditions to the bacteriophage exposed sample sufficient to: allow the bacteriophage to infect the target microorganism, and multiply in the target microorganism to create progeny bacteriophage; and produce a dissociated bacteriophage substance accessible to an assay; and assaying the bacteriophage exposed sample to determine the presence or absence of the bacteriophage substance as an indication of the presence or absence of the target microorganism in the sample. In the above summary, it should be understood that a bacteriophage substance can be both a dissociated bacteriophage substance and at the same time be associated with the bacteriophage.

In another preferred embodiment, the invention provides a method of detecting the presence or absence of microorganism in a sample to be tested, the method comprising: combining with the sample, parent bacteriophage capable of infecting the target microorganism to create a bacteriophage exposed sample; (b) providing conditions to the bacteriophage exposed sample sufficient to: allow the bacteriophage to infect the target microorganism and multiply in the target microorganism to create a detectable amount of either the bacteriophage or a biological substance associated with the bacteriophage in the bacteriophage exposed sample; (c) actively lysing the microorganism; and (d) assaying the bacteriophage exposed sample to detect the presence or absence of the bacteriophage or the biological substance associated with the bacteriophage to determine the presence or absence of the target microorganism. Preferably, the actively lysing comprises adding a microbial lysozyme to the bacteriophage exposed sample. Preferably, the actively lysing comprises a method selected from the group consisting of: adding chloroform to the bacteriophage exposed sample; treating the bacteriophage exposed sample with acid; and physically processing the bacteriophage exposed sample.

The invention also provides apparati and methods for detecting microorganisms in which amplified phage induce a color change in a substrate. In the preferred embodiment, the invention provides apparatus for detecting a target microorganism, the apparatus comprising: a substrate; an immobilization zone on the substrate, the immobilization zone including an immobilization agent designed to immobilize a bacteriophage or a biological substance associated with a bacteriophage; and a color moderator designed to interact with a bacteriophage or a biological substance associated with a bacteriophage, whereby the presence of the bacteriophage or the biological substance associated with a bacteriophage causes the immobilization zone to change color. Preferably, the immobilization zone comprises antibodies. Preferably, the color moderator comprises colored beads. In another embodiment, the color moderator comprises a reacting agent and an enzyme which form a precipitant upon reacting. In the preferred embodiment of the corresponding method, a bacteriophage exposed sample is applied to a substrate at least a portion of which changes color if either the bacteriophage or a biological substance associated with the bacteriophage in said bacteriophage exposed sample is present.

The invention also provides a kit for determining the presence or absence of a target microorganism in a sample to be tested, the kit comprising: a first container containing a bacteriophage capable of infecting the target microorganism; and a substrate at least a portion of which changes color if either the bacteriophage or a biological substance associated with the bacteriophage in the bacteriophage exposed sample is present. Preferably, the kit further comprises a second container containing a buffer solution. Preferably, the substrate comprises a lateral flow strip or a SILAS surface. Preferably, the first container includes a dropper designed to release drops of a predetermined size.

The invention also provides a method of manufacturing a microbial, preferably a bacterial, test substrate, the method comprising: providing a substrate and a biological material capable of attaching to a bacteriophage or a biological substance associated with the bacteriophage; forming a line of the biological material on the substrate; and cutting the substrate in a direction essentially perpendicular to the line to form the test substrate. Preferably, the substrate is a porous membrane. Preferably, the biological material is an antibody.

It is an object of the invention to provide a phage amplification method of detecting microorganisms in which the parent phage are not destroyed, removed, neutralized, or inactivated in the bacteriophage exposed sample.

It is an object of the invention to provide a highly specific bacterium detection method.

It is a further object of the invention to provide a broad-spectrum bacterium detection method.

It is another object of the invention to provide a bacterium detection method that can be used to detect bacteria in low concentrations.

It is still another object of the invention to provide a bacterium detection method that can detect bacteria over a wide range of concentrations.

It is a further object of the invention to provide a bacterium detection method that gives rapid results as compared to most existing detection methods.

It is yet another object of the invention to provide a bacterium detection method that is inexpensive relative to existing bacteria detection methods.

It is still another object of the invention to provide a bacterium detection method that is simple to perform and does not require highly skilled technicians or complex instrumentation.

It is yet a further object of the invention to provide a bacterium detection method that can be performed in the field or at point of care.

It is still a further object of the invention to provide a bacterium detection method that is readily multiplexed such that multiple bacteria are detected in a test sample.

It is yet another object of the invention to provide a bacterium detection method that uses the detection of a specific phage biomarker as a surrogate for detecting target bacteria present in a sample.

It is still another object of the invention to provide a bacterium detection method that utilizes highly specific phage infection of target bacteria as a means of specifically detecting the presence of said target bacteria in a sample.

It is another object of the invention to provide a bacterium detection method that can be used to detect any bacteria for which a suitable phage exists.

It is a further object of the invention to provide a bacterium detection method that utilizes genetically unmodified phage.

It is another object of the invention to provide a microorganism detection method which utilizes a genetically modified bacteriophage. For example, the bacteriophage can be genetically modified to enhance a desirable property of the infection process, to over-express a detectable biomarker, to express an enzyme, or to express a target on the capsid protein.

It is yet a further object of the invention to provide a bacterium detection method that uses phage amplification as a means of achieving high sensitivity.

It is still a further object of the invention to provide a bacterium detection method wherein the phage is detected by detecting a specific biomarker associated with the phage, such as the capsid sheath of the phage.

It is yet another object of the invention to provide a bacterium detection method wherein the phage biomarkers are individual, dissociated proteins from the phage.

It is a further object of the invention to provide a bacterium detection method wherein the phage biomarkers are phage nucleic acids.

It is a further object of the invention to provide a bacterium detection method utilizing a second amplification process consisting of dissociating the phage prior to the detection process.

It is another object of the invention to provide a bacterium detection method that utilizes tagged parent phage that can be distinguished from progeny phage that are produced from phage amplification.

It is still a further object of the invention to provide a bacterium detection method that uses antibodies to bind to a phage biomarker producing an antibody-phage complex.

It is yet another object of the invention to provide a bacterium detection method using existing immunoassay techniques to detect specific antibody-antigen binding events as a means of detecting antibody-phage complexes and thereby detecting the presence of target bacteria in a sample.

It still another object of the invention to provide a bacterium detection method that utilizes lateral flow strips to detect phage and thereby detecting the presence of target bacteria in a sample.

It is yet another object of the invention to provide a method for detecting antibiotic resistant strains of bacteria.

The above summary is intended to illustrate some examples of the objects, features, and advantages of the invention so that the invention can be better understood. In some embodiments of the invention, only one of the above objects may be realized, and in others a plurality of such objects may be realized. However, the above objects are intended to be exemplary, not all inclusive, so there will be instances in which none of the above objects are realized in a particular embodiment. For example, the methods and apparati of the invention can be used for detecting microorganisms other than bacteria, such as fungi, mycoplasmas, protozoa, and other microscopic living organisms. Thus, if the word “bacteria” in the above objects is replaced with the more general term “microorganism”, valid objects of the invention are expressed. Numerous other features, objects, and advantages of the invention will become apparent from the following description when read in conjunction with the accompanying drawings.

### BRIEF DESCRIPTION OF THE DRAWINGS

FIG. 1 illustrates a first embodiment of the invention wherein phage are added to the sample to give an initial concentration below the detection limit;

FIG. 2 illustrates the incubation process of phage infection, amplification, and cell lysis;

FIGS. 3A, 3B, and 3C illustrate the usage of a lateral flow device to detect phage in a test sample;

FIG. 4 is a side cross-sectional view of the flow device of FIG. 3A;

FIG. 5 is an illustration of a bacteriophage;

FIG. 6 illustrates a second embodiment of the invention wherein phage are added to the sample to give an initial concentration below the detection limit and where the phage are dissociated such that phage subcomponent biomarkers are detected;

FIG. 7 illustrates a third embodiment wherein tagged phages are added to the sample;

FIG. 8 illustrates a fourth embodiment of the invention wherein tagged parent phage are added to the sample and where the progeny phage are dissociated such that phage subcomponent biomarkers are detected;

FIG. 9 illustrates detection of antibiotic resistant bacteria using the invention;

FIG. 10 illustrates a phage amplification process;

FIG. 11 is a MALDI spectrum showing mass versus intensity for the bacteriophage T4 illustrating some possible biomarkers;

FIGS. 12 through 16 illustrate a phage-based assay process using a SILAS surface;

FIG. 17 illustrates a negative result from the assay of FIGS. 12-16;

FIG. 18 illustrates a positive result from the assay of FIGS. 12-16;

FIG. 19 shows a test kit according to the invention;

FIG. 20 shows exemplary directions for using the test kit of FIG. 19 and illustrates the use of the test kit;

FIG. 21 illustrates an exemplary assay according to the invention utilizing a bacteriophage genetically modified to enhance a desirable property of the infection process;

FIG. 22 illustrates an exemplary assay according to the invention utilizing a bacteriophage genetically modified to over-express a detectable biomarker;

FIG. 23 illustrates an exemplary assay according to the invention utilizing a bacteriophage genetically modified to express an enzyme;

FIG. 24 illustrates an exemplary assay according to the invention utilizing a bacteriophage genetically modified to express a target on the capsid protein;

FIG. 25 shows an exemplary MALDI spectrum of an MS2-E. coli bacteriophage infected sample at a time immediately after introduction of the bacteriophage;

FIG. 26 shows an exemplary MALDI spectrum of an MS2-E. coli bacteriophage infected sample at a time 30 minutes after the time of the spectrum of FIG. 24;

FIG. 27 is a block diagrammatic illustration of a MALDI mass spectrometer; and

FIG. 28 illustrates an exemplary embodiment of an assay process according to the invention utilizing immunomagnetic separation and a MALDI mass spectrometer.

### DETAILED DESCRIPTION OF THE INVENTION

The method of the invention relies on the usage of bacteriophage, or simply phage, to detect the presence of target microscopic living organism (microorganism), such as a bacterium, in a sample. In this disclosure, the terms “bacteriophage” and “phage” include bacteriophage, phage, mycobacteriophage (such as for TB and paraTB), mycophage (such as for fungi), mycoplasma phage or mycoplasmal phage, and any other term that refers to a virus that can invade living bacteria, fungi, mycoplasmas, protozoa, and other microscopic living organisms and uses them to replicate itself. Here, “microscopic” means that the largest dimension is one millimeter or less. Bacteriophage are viruses that have evolved in nature to use bacteria as a means of replicating themselves. A phage does this by attaching itself to a bacterium and injecting its DNA into that bacterium, inducing it to replicate the phage hundreds or even thousands of times. Some bacteriophage, called lytic bacteriophage, rupture the host bacterium, releasing the progeny phage into the environment to seek out other bacteria. The total incubation time for phage infection of a bacterium, phage multiplication or amplification in the bacterium, to lysing of the bacterium takes anywhere from tens of minutes to hours, depending on the phage and bacterium in question and the environmental conditions.

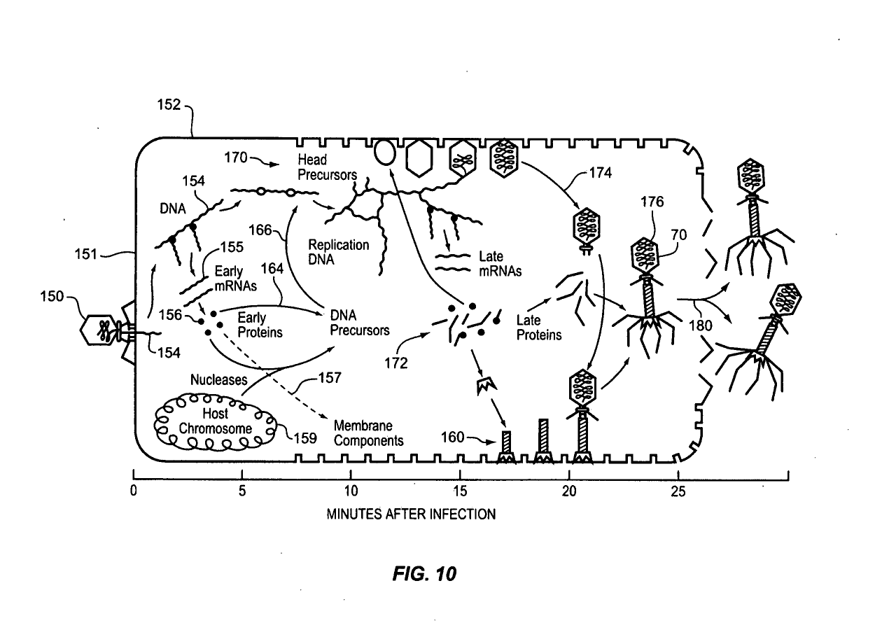

The disclosed detection method offers a combination of specificity, sensitivity, simplicity, speed, and/or cost which is superior to any currently known microscopic organism detection method. The method taught herein relies on the usage of bacteriophage to indirectly detect the presence of one or more target bacterium in a sample. A typical bacteriophage 70, in this case MS2-E. Coli, is shown in FIG. 5. Structurally, a bacteriophage 70 comprises a protein shell or capsid 72, sometimes referred to as a head, that encapsulates the viral nucleic acids 74, i.e., the DNA and/or RNA. A bacteriophage may also include internal proteins 75, a neck 76, a tail sheath 77, tail fibers 78, an end plate 79, and pins 80. The capsid 72 is constructed from repeating copies of one or more proteins. Referring to FIG. 10, when a phage 150 infects a bacterium 152, it attaches itself to a particular site on the bacterial wall or membrane 151 and injects its nucleic acid 154 into that bacterium, inducing it to replicate the phage from tens to thousands of copies. The process is shown in schematic in FIG. 10. The DNA evolves to early mRNAS 155 and early proteins 156, some of which become membrane components along line 157 and others of which utilize bacteria nucleases from host chromosomes 159 to become DNA precursors along line 164. Others migrate along the direction 170 to become head precursors that incorporate the DNA along line 166. The membrane components evolve along the path 160 to form the sheath, end plate, and pins. Other proteins evolve along path 172 to form the tail fibers. When formed, the head releases from the membrane 151 and joins the tail sheath along path 174, and then the tail sheath and head join the tail fibers at 176 to form the bacteriophage 70. Some bacteriophage, called lytic bacteriophage, rupture the host bacterium, shown at 180, releasing the progeny phage into the environment to seek out other bacteria. Lytic phages are typically used in the method disclosed herein. However, non-lytic phages can be used, particularly if they or the bacteria can be activated to release progeny phage or portions of progeny phage after the progeny phage infect the host bacteria.

The total cycle time for phage infection of a bacterium, phage multiplication or amplification in the bacterium, to lysing of the bacterium takes anywhere from minutes to hours, depending on the phage and bacterium in question and the environmental conditions. As an example, the MS2 bacteriophage infects strains of Escherichia coli and is able to produce 10,000 copies to 20,000 copies of itself within 40 minutes after attachment to the target cell. The capsid of the MS2 phage comprises 180 copies of an identical protein. This means that for each E. coli infected by MS2, upwards of 1.8×10⁶ individual capsid proteins are produced. The process of phage infection whereby a large number of phage and an even larger number of capsid proteins are produced for each infection event is called phage amplification.

Microbiologists have isolated and characterized many thousands of phage species, including specific phages for most human bacterial pathogens. Individual bacteriophage species exist that infect bacterial families, individual species, or even specific strains. Table 1 lists some such phages and the bacterium they infect.

TABLE 1

| PHAGE | BACTERIAL TARGET |
|---|---|
| MS2 | E. coli, Enterococci |
| φA1122 | Yersinia pestis |
| CSLEC0157 | E. coli 0157 |
| φFelix 0-1 | Salmonella spp. |
| Chp1 | Chlamydia trachomatis |
| Gamma | B. anthracis |
| A511 | Listeria spp. |

This invention takes advantage of the existing characteristics of bacteriophage, such as highly specific phage-bacterial infection, phage amplification, and short incubation time, resulting in a bacterial detection method which is highly specific to target bacteria, very sensitive, fast, simple to perform, and/or can be quite economical. Moreover, unlike other phage-based bacterial detection methods, the preferred method described herein uses phages that are not genetically modified to include bioreporter or inducer genes. This dramatically reduces the time and costs associated with developing specific bacterial tests utilizing this method.

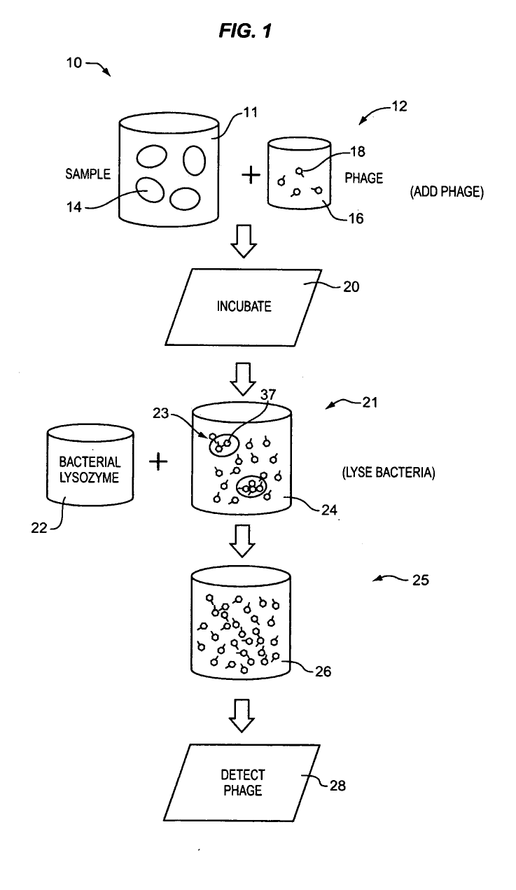

FIG. 1 illustrates a first embodiment 10 of the method to detect specific bacteria in a sample. In a first ‘ADD PHAGE’ process 12, parent bacteriophage 18 that will infect the target bacterium 14 is combined with the raw sample 11 of bacterium 14. In the preferred embodiment, the bacteriophage, preferably in a suspension or solution 16, is added in a predetermined concentration to the raw sample II of bacterium 14. Here, the term “raw sample” refers to the sample prior to the addition of the phage. The raw sample/phage combination is referred to herein as “the test sample 24” or the “bacteriophage exposed sample 24”. If the object of the method is to detect a specific bacterium at the species or strain level, then a correspondingly specific phage is used in the method. For example, the φA1122 phage can be used to specifically detect Y. Pestis. Conversely, a less specific phage can be used to detect a wider range of bacteria in a sample. The phage MS2 will infect many different E. coli species as well as Enterococci and, thus, is quite suitable for detecting fecal contamination in water.

To detect multiple bacteria, one species of bacteriophage is added to the raw sample for each target bacterium giving a single test sample that contains all of the target bacteria and associated phages. For the purposes of simplicity, the method will be described henceforth as it applies to detecting a single bacterium. It should be clear to those skilled in the art how each process of the method can be performed simultaneously with one test sample utilizing unique bacterium/phage combinations to detect each target bacterium.

The raw sample 11 containing the target bacterium 14 is generally in a liquid form but could be a solid or a powder. The raw sample could be a mixture or suspension containing many different organic and inorganic compounds. It may have been pretreated in a variety of ways to prepare it for testing. For example, the raw sample may have been purified or filtered to remove unwanted components or to concentrate the target bacterium. It may have been cultured in a media conducive to the incubation of the target bacterium or to induce the target bacterium into a more viable state. The raw sample may be in a relatively untreated state such as might be the case with a sputum, blood, or water sample. It should be clear to one skilled in the art that pretest sample preparation may include any one of a wide variety of suitable processes and the raw sample may take many different forms.

The phage itself may be added to the sample in a variety of forms. It may be added in a dry state. The phage may be mixed or suspended into a liquid reagent mixture. It may be suspended in a vial to which the raw sample is added. It also may take any other suitable form. The phage added to the raw sample is herein referred to as “the parent phage”.

Returning to FIG. 1, the test sample 24 is incubated, preferably for a predetermined time. For this method, the test sample should preferably be in a condition that is conducive to phage infection of the target bacterium prior to the incubation process. This can be accomplished in a variety of ways well known to those skilled in the art. For example, the parent phage may be mixed into a reagent that, when added to the raw sample, results in a test sample conducive to infection. The test sample may be prepared in many different ways to establish conditions conducive to phage infection.

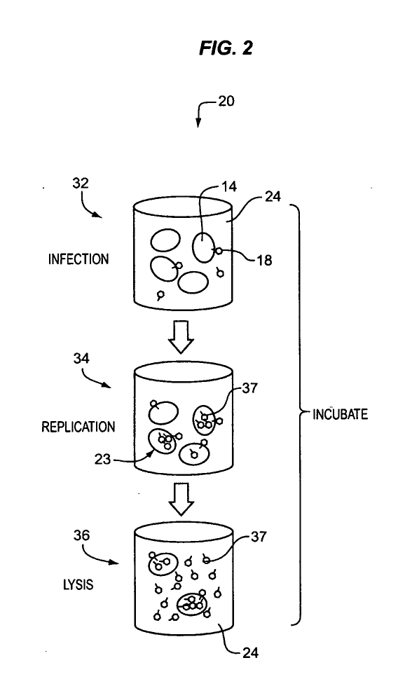

The INCUBATE process 20 is shown in FIG. 2. The parent phage 18 infects 32 the target bacteria 14 by attaching themselves to cell walls of the target bacteria and injecting the viral nucleic acid to create infected bacteria 23. Replication 34 of progeny phage as indicated in FIG. 10 then proceeds within the host bacteria. If lytic phages are used, the host ruptures in a lysis process 36 releasing the progeny phage 37 into the test sample where they may infect other target bacteria. This incubation process may proceed for one or more cycles of infection, amplification, and lysis. Assuming there were target bacteria in the raw sample, the test sample will contain a large number of progeny phage for each individual bacterium infected during the incubation process.

At the conclusion of the INCUBATE process, some of the infected target bacteria may not have lysed. Under these circumstances, many or even all of the progeny phage will still be held within host bacteria and as such may not be directly detectable. To address this potential problem, an optional process 21 and 25 LYSE BACTERIA is accomplished as shown in FIG. 1 by adding a microbial lysozyme 22 for the particular microorganism to the test sample at 21, which, in process 25, causes the cell walls of essentially all the particular microorganism, such as a bacterium, present in the test sample 24 to rupture, thereby releasing essentially all progeny phage contained therein. When necessary to distinguish the LYSE step from the natural or passive lysing caused by the bacteriophage, we will refer to it as “actively lysing” herein. The usage of the microbial, or more specifically, bacterial, lysozyme can shorten the time required to carry out the method taught herein because one need not wait for all of the target bacteria to lyse on their own. For slowly incubating phage, this can make a substantial difference. For the purposes of this invention, the term “actively lysing” shall refer to any material, apparatus, or process by which the microorganism host is induced to rupture, thus releasing the progeny phage into the test sample, including, but not limited to, chemical means such as traditional lysozymes, chloroform, or acid treatments or a physical process, such as changing the osmotic pressure.

Process 28, DETECT PHAGE, of the embodiment illustrated in FIG. 1 comprises detecting a biomarker associated with the phage. If this biomarker is detected, it is an indirect indicator of the presence of the target bacteria in the raw sample. For the embodiment of the method described thus far, the parent phage added to the raw sample and the progeny phage, if produced during the incubation process, are identical. This means that, even if there are no target bacteria in the test sample, there will still be phage present during detection process 28 that could give rise to an associated background signal. A method of solving this problem is to control the initial concentration of parent phage in the test sample such that the background signal they produce is undetectable in detection process 28. Thus, if no target bacteria are present in the test sample such that no phage amplification occurs, then no signal is detected at the end of the test. A higher concentration of parent phage can still be used, provided that the background signal can be distinguished from the signal arising from the parent plus progeny phage. The lowest bacterial concentration at which the resulting signal in process 28 can be distinguished from the background signal represents the sensitivity limit of the method. A simple way of doing this is to perform a test on a reference sample in which it is known that no bacteria are present. This creates a reference result which can be compared with a corresponding result from a test sample. If the test result from the test sample clearly shows a higher signal level than the reference sample, then the presence of the target bacteria is indicated.

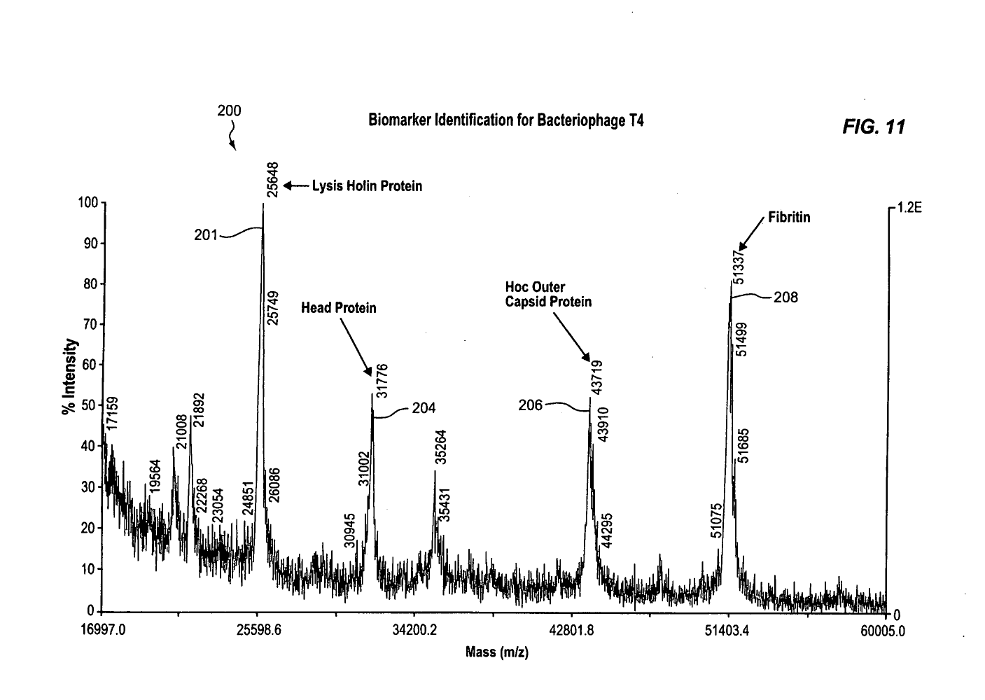

For the purposes of this invention, any biomarker associated with the phage may be used as an indirect detection means of the target bacteria in the raw sample. This can include any portion of the phage shown in FIGS. 5 and 10. For example, FIG. 11 shows a MALDI spectrum 200 graphing percent intensity versus mass for a bacteriophage T4. The spectrum 200 shows significant peaks 201 for the lysis holin protein, 204 for the head protein, 206 for the hoc outer capsid protein, and 208 for the fibritin. These large peaks indicate that any of these phage portions can be used as a biomarker in the MALDI detection method, which will be discussed below. A very useful phage biomarker is the phage capsid 72. The capsid comprises many copies, often in excess of one hundred, of one or more proteins. Thus, there are multiple, identical binding sites around each individual phage particle that can be used for detection purposes. This increases the potential signal level deriving from each phage particle contained in the test sample. The advantage of detecting phage or a phage-associated biomarker as opposed to directly detecting a biomarker associated with lysed target bacteria is dramatically increased sensitivity. The concentration of the phage in the incubated test sample is much higher than that of the target bacteria in the raw sample because of phage amplification. Phage amplification can boost the signal associated with each single bacterium present in the raw sample by many orders of magnitude, i.e., factors of ten. Therefore, lower concentrations of bacteria can be detected with this method than with other methods that do not utilize a phage amplification process.

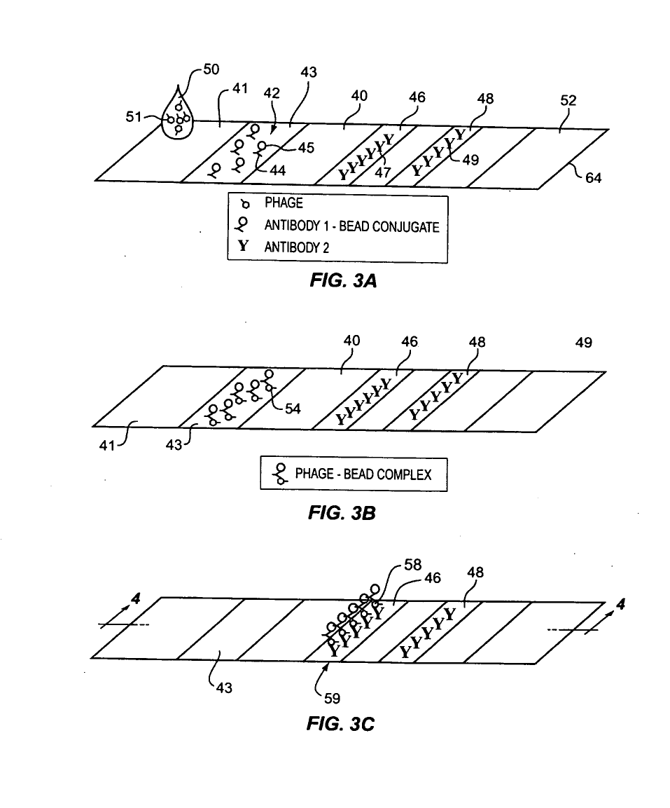

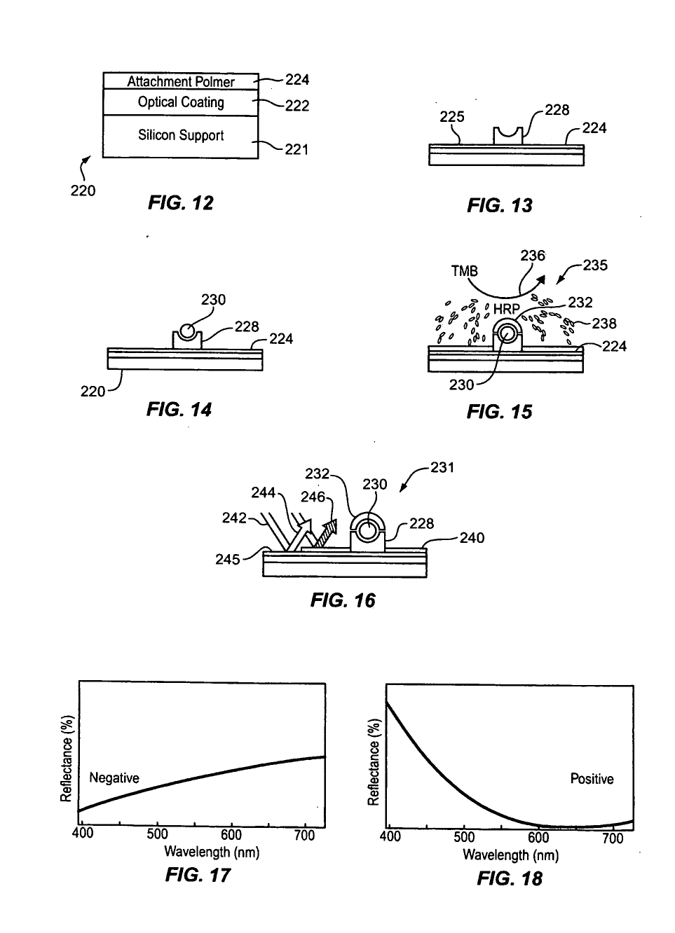

Any detection method or apparatus that detects some biomarker associated with the phage will suffice for this method 28. Preferred methods are immunoassay methods utilizing antibody-binding events to produce detectable signals including ELISA, flow cytometry, western blots, aptamer-based assays, radioimmunoassay, immunoflouresence, and lateral flow immunochromatography (LFI). Other methods are matrix-assisted laser desorption/ionization with time-of-flight mass spectrometry (MALDI-TOF-MS), referred to herein as MALDI, and the use of a SILAS surface which changes color as a detection indicator. One immunoassay method, LFI, is discussed in detail below in connection with FIGS. 3, 5, and 19; the SILAS color indicator method is discussed in detail below in connection with FIGS. 12-18; and the MALDI method is discussed in detail below in connection with FIGS. 11 and 24-27.

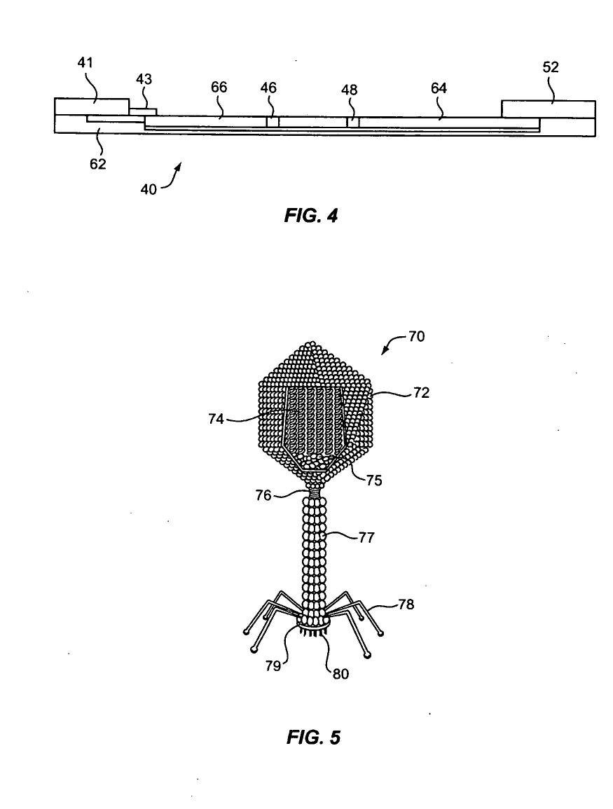

FIG. 3 illustrates how LFI can be implemented with a lateral flow strip 40 to detect the presence of phage in a test sample. A cross-sectional view of the lateral flow strip 40 is shown in FIG. 4. The lateral flow strip 40 preferably includes a sample application pad 41, a conjugate pad 43, a substrate 64 in which a detection line 46 and an internal control line 48 are formed, and an absorbent pad 52, all mounted on a backing 62, which preferably is plastic. The substrate 64 is preferably a porous mesh or membrane. It is made by forming lines 43, 46, and optionally line 48, on a long sheet of said substrate, then cutting the substrate in a direction perpendicular to the lines to form a plurality of substrates 64. The conjugate pad 43 contains colored beads 45 each of which has been conjugated to a first antibody 44, referred to herein as first antibody, forming first antibody-bead conjugates 42. First antibody 44 selectively binds to the phage 51 in the test sample. Detection line 46 and control line 48 are both reagent lines and each form an immobilization zone; that is, they contain a material that interacts in an appropriate way with the bacteriophage or other biological marker. In the preferred embodiment, the interaction is one that immobilizes the bacteriophage or other biological marker. Detection line 46 comprises immobilized second antibodies 47, with antibody line 46 perpendicular to the direction of flow along the strip, and being dense enough to capture a significant portion of the phage in the flow. Second antibody 47 also binds specifically to the phage 51. First antibody 44 and second antibody 47 may or may not be identical. Either may be polyclonal or monoclonal antibodies. Optionally, strip 40 may include a second reagent line 48 including a third antibody 49. Third antibody 49 may or may not be identical to one or more of the first and second antibodies. Second reagent line 48 may serve as an internal control zone to test if the assay functioned properly.

One or more drops of a test sample 50 are added to the sample pad as shown in FIG. 3A. The test sample 50 preferably contains parent phage as well as progeny phage if the target bacterium was present in the original raw sample. The test sample flows along the lateral flow strip 40 toward the absorbent pad 52 at the opposite end of the strip. As the phage particles flow along the conjugate pad toward the membrane, they pick up one or more of the first antibody-bead conjugates 42 forming phage-bead complexes 54 as shown in FIG. 3B. As the phage-bead complexes move over row 46 of second antibodies 47, they form an immobilized and concentrated first antibody-bead-phage-second antibody complex 58 as shown in FIG. 3C. If enough phage-bead complexes bind to the row 46 of immobilized second antibodies, a colored line 59 becomes visible to the naked eye. A visible line 59 indicates that the target bacteria were present in the raw sample. If no line is formed, then target bacteria were not present in the raw sample or were present in concentrations too low to be detected with the lateral flow strip 40. For this test to work reliably, the concentration of parent phage added to the raw sample should be low enough such that the parent phage alone are not numerous enough to produce a visible line on the lateral flow strip. The antibody-bead conjugates 45 are color moderators that are designed to interact with the bacteriophage or a biological substance associated with the bacteriophage. When they are immobilized in the immobilization zone 46, they cause the immobilization zone to change color.

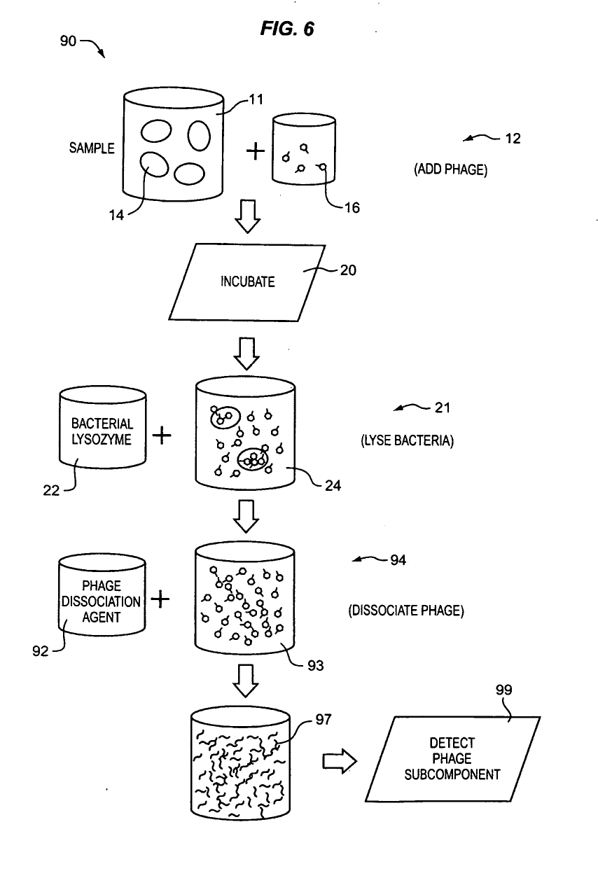

FIG. 6 illustrates a second embodiment 90 of a method to detect target bacteria according to the invention, which method 90 has enhanced sensitivity. Processes 12, 20, and optional process 21 consisting of ADD PHAGE, INCUBATE, and LYSE BACTERIA are identical to the corresponding processes described in association with FIG. 1. After optional process 21, the test sample contains an abundance of phage particles if target bacteria were present in the raw sample.

As shown in FIG. 6, process 94 of the second embodiment 90, DISSOCIATE PHAGE, comprises adding a phage dissociation agent 92 to the test sample. The phage dissociation agent 92 breaks up the phage particles into their constituent components 97 including individual capsid proteins and viral nucleic acids. Examples of phage dissociation agents are acid treatments, urea, denaturing agents, and enzymes. Any suitable phage dissociation agent may be used. In this process, a dissociated bacteriophage substance 97 is produced.

Process 99 of the embodiment illustrated in FIG. 6, DETECT PHAGE SUBCOMPONENT, comprises detecting a biomarker, i.e., dissociated bacteriophage substance 97, associated with the dissociated phage subcomponents. With respect to the foregoing discussion, it should be understood that a bacteriophage substance can be both a dissociated bacteriophage substance and at the same time be associated with the bacteriophage. That is, the phrase “a dissociated bacteriophage substance” means a substance that is no longer a part of a whole bacteriophage, while the term “associated with the bacteriophage” means that substance was at one time a part of a bacteriophage or is produced in the process of bacteriophage replication. Owing to the usage of the phage dissociation agent in process 94, there are an abundance of individual capsid proteins 97 that can be detected in process 99. As with the first embodiment, these can be detected using established antigen-antibody based immunoassay techniques. In addition, the exposed viral genetic material can be detected with other established techniques including PCR, genetic probe biosensors, photoaptamers, molecular beacons, or gel electrophoresis. Any appropriate phage biomarker detection method or apparatus may be used.

Keeping the concentration of parent phage in the test sample below the background detection limit makes for a very simple test method: add phage to the raw sample, incubate, and then detect phage biomarkers. However, there is a potential disadvantage as well. The potentially low concentration of parent phage may result in conditions where the ratio of parent phage to target bacteria in the test sample is less than 1; i.e., the Multiplicity Of Infection (MOI) is low. To ensure that all target bacteria in the test sample have a high probability of being infected, the incubation time in Process 20 can be made be made longer, for example, a time equivalent to two or more cycles of infection and lyses. Thus, test simplicity is offset by potentially longer testing times. This potential limitation can be overcome if the signal associated with the parent phage can be eliminated or significantly reduced such that higher concentrations of parent phage can be utilized—MOIs greater than 5. It can also be overcome if the signal due to the progeny phage is enhanced, such as by the use of the capsid protein as a biological marker or by the use of genetically enhanced phage, both of which are discussed in detail herein.

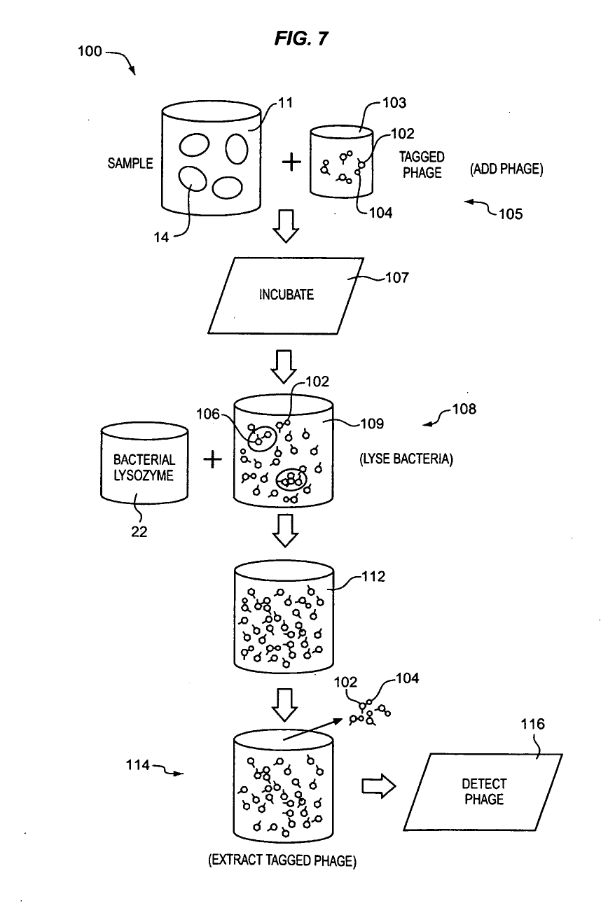

FIG. 7 illustrates a third embodiment 100 of the inventive method to detect target bacteria in a sample wherein more rapid results are achievable. In this embodiment of the invention, the parent phage 102 that are combined with the raw sample are tagged, indicated by a tag symbol at 104, such that they can be subsequently removed from the test sample, isolated from the portion of the test sample in which the bacteriophage are detected, or otherwise neutralized prior to analysis such that primarily untagged, progeny phage contribute to the detected signal. For example, in one embodiment a biotinylated phage was used as a parent phage. Biotinylated bacteriophage are strongly attracted to streptavidin. This strong affinity was used to subsequently segregate the tagged parent phage from the test sample, as discussed below. The tagged parent phage can also be attached to a physical substrate, such as by coating a probe or mesh structure with the parent bacteriophage or by chemically binding the phage to the substrate. The tagged bacteriophage then can be segregated from the progeny bacteriophage by removing the substrate from the test sample or by detecting the bacteriophage in a portion of the test sample that is segregated from the substrate.

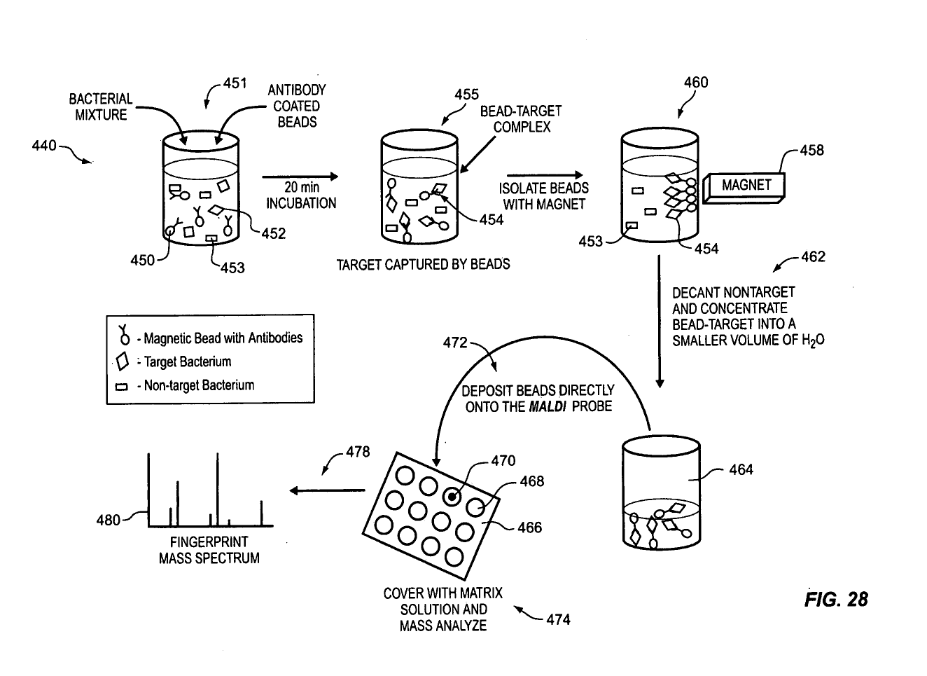

Processes 105, 107, and 108, ADD PHAGE, INCUBATE, and LYSE BACTERIA, respectively, of this embodiment are the same as processes 12, 20, and 21, respectively, of FIGS. 1 and 6, with the exception that the solution of parent phage 103 added to the raw sample in Process 105 contains tagged phage 102 so bacteriophage exposed sample 109 contains both tagged phage 102 external of the bacteria and untagged progeny phage 106 within the bacteria. Thus, the lysed solution 112 will contain both tagged and untagged phage. In process 114, EXTRACT TAGGED PHAGE, the tagged parent phage are segregated from the progeny bacteriophage by extracting or substantially removing them from the test sample or otherwise isolating the parent phage from the progeny phage such that they do not contribute to the analyzed signal. If the tagged parent phages are attached to a physical substrate when added to the raw sample in process 105, then the substrate and associated parent phage are preferably physically removed from the test sample in process 114. Biotinylated phage that are not attached to a physical substrate also can be readily segregated or removed from the test sample. In one embodiment, streptavidin-coated magnetic beads were added to the test sample where they rapidly collected the biotinylated parent phage. A magnet was then used to aggregate and remove the magnetic beads along with the bound parent phage from the test sample. See the discussion of magnetic extraction associated with FIG. 28 below. Similarly, in another embodiment, a streptavidin-coated mesh was stirred through the test sample, gathering up essentially all of the biotinylated parent phage from the test sample. Other physical substrates or probes other than a mesh can also be used. In another embodiment, a lateral flow device was used. A portion 66 (FIG. 4) of the mesh substrate 64 prior to the antibody strip 46 was impregnated with streptavidin, coating the mesh fibers. The streptavidin-coated mesh gathered up and immobilized the tagged parent phage by binding the parent phage to the portion 66 before they reached the antibody strip 46. The progeny phage did not bind to the streptavidin and, thus, flowed freely down the strip and were visually detected. Similarly, other portions of the lateral flow device could be coated or impregnated with streptavidin, such as the sample pad 41 onto which the test sample is dropped.

The method described herein is not limited to these examples of tagging parent phage and subsequently removing them from or segregating them within the test sample. Other parent phage tagging/phage segregation methods will be readily apparent to those skilled in the art.

Process 116 of the embodiment illustrated in FIG. 7, DETECT PHAGE, is to analyze the test sample to detect a biomarker associated with the progeny phage as a surrogate marker for target bacteria present in the raw sample. The detection means used with this embodiment are identical to those described with respect to processes 28 and 29 of the embodiments 10 and 90, respectively, as illustrated in FIGS. 1 and 6, respectively. As with the earlier embodiments, any suitable detection method or apparatus may be used.

FIG. 8 illustrates a fourth embodiment 120 of a method to detect target bacteria in a sample, in which method 120 more rapid results are achievable and the sensitivity is enhanced. Embodiment 120 is a combination of the methods taught in embodiments 90 and 100. Processes 105, 107, 108, and 114 are identical to those taught with embodiment 100 and illustrated in FIG. 7, i.e., ADD PHAGE, INCUBATE, optionally LYSE BACTERIA, and EXTRACT TAGGED PHAGE, respectively. Specifically, embodiment 120 incorporates tagged parent phage in process 105 and a parent phage removal or segregation process in process 114.

In process 121, DISSOCIATE PHAGE, a phage dissociation agent 122 is added to the test sample 124 as taught in process 94 of embodiment 90 and illustrated in FIG. 6. In a preferred embodiment, the tagged parent phage is physically removed from the test sample in process 114 rather than simply segregated so that it will not be exposed to the phage dissociation agent in process 121. Thus, the test sample 124 contains only progeny phage, and the dissociated test sample 126 will contain biological marker material, such as capsid proteins 128, only from progeny phage. In this manner, the amplification associated with dissociating the phage capsid proteins 128 will combine with the phage amplification of process 107, resulting in a much higher total amplification. For example, if the phage amplification process gives an amplification of 1000 per bacterium and the phage has 100 copies of a particular capsid protein, then the combined amplification will be 10³×10² or 10⁵ per target bacteria infected in the test sample. If the parent phage is not removed, then the total amplification is only the phage amplification that occurs in process 107; i.e. 10³, because the amplification arising from dissociating the phage will occur to both the parent phage and to the progeny phage, thus canceling out the second amplification process.

DETECT PHAGE SUBCOMPONENT process 130 of the embodiment 120 illustrated in FIG. 6 is preferably the same as any of the processes 28, 99, and 116 of the earlier embodiments.

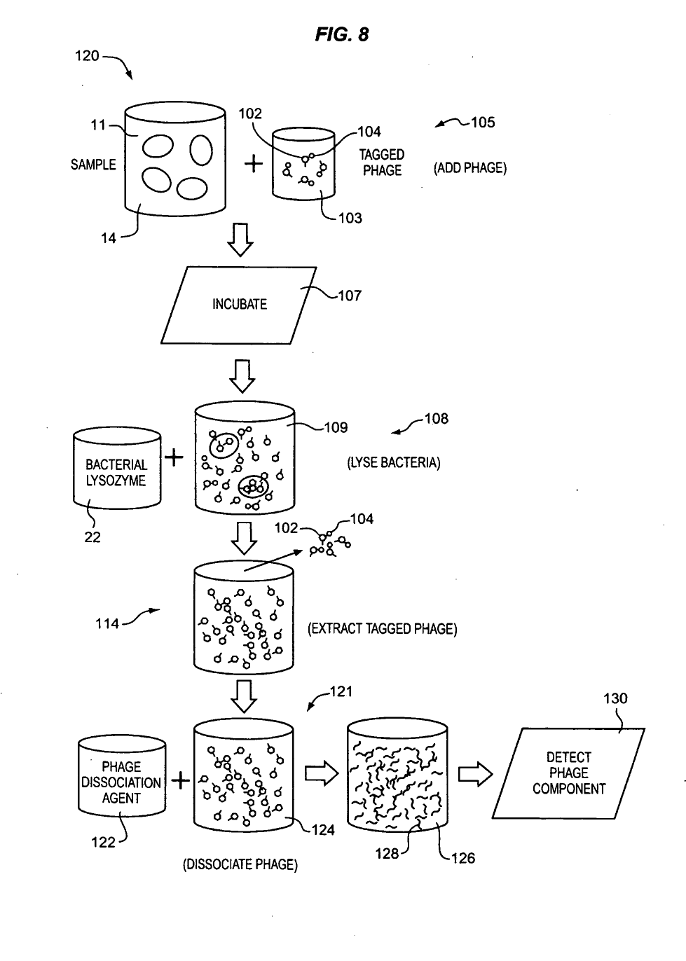

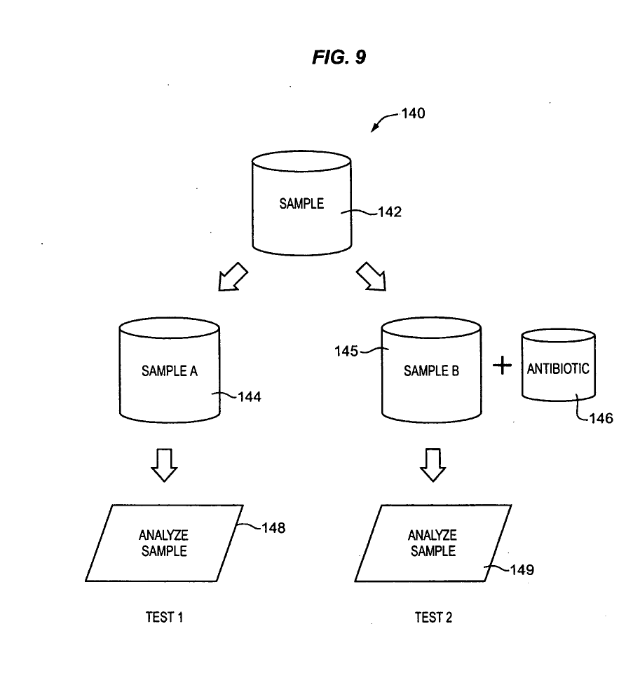

FIG. 9 illustrates a method 140 by which any of the embodiments of the invention can be used to detect a target bacterium, and if present, determine if it is resistant to one or more antibiotics. A sample 142 that may contain the target bacterium is divided into two, a first Sample A, indicated by 144, and a second Sample B, indicted by 145. A first antibiotic 146 is added to Sample B whereupon the target bacteria in Sample B are killed if they are not resistant to the first antibiotic. Samples A and B are then analyzed at 148 and 149 to detect the presence of viable target bacteria in each, giving Result A and Result B. Any of the methods taught in this invention can be used for these analyses. If Result A is positive, it indicates that the target bacterium is present in the original sample. If Result B is also positive, it indicates that the target bacterium is resistant to the first antibiotic. If, on the other hand, Result B is negative, then the target bacterium is not resistant to the first antibiotic. To screen for antibiotic resistance to any one of a range of antibiotics simultaneously, then all of the antibiotics of interest are added to Sample B prior to analyzing for the target bacterium. If the target bacterium is detected in both the pure sample and the antibiotic treated sample, it indicates that the target bacterium in the sample is resistant to one of the added antibiotics. This process can also be used to determine the susceptibility of bacteria to antibiotics or other decontaminants. It can also be used to test whether a bacterial decontamination process has been successful. By dividing a sample into a control portion and a test portion, the effectiveness of bacteriological methods and materials can be tested. Those skilled in the art will recognize that the processes of the invention can be used in nearly every instance where it is desirable to determine if live bacteria are present.

FIGS. 12 through 18 illustrate another embodiment of detection processes 28, 99, 116, and 130. This process uses a SILAS surface 220. A SILAS surface 220 comprises a semiconducting or insulating wafer 221 having an optical coating 222 covered with an attachment polymer 224. As known in the art, the SILAS surface is designed to reflect specific wavelengths of light and to attenuate others by interference. These surfaces generate a visible signal by the direct interaction of light with the thin films formed on the surface. The thin films include optical coatings and/or biological films created by binding of specific target molecules to the surface. A positive result is usually seen as a color change from gold to purple because the optical path of the light is lengthened by the accumulated biological mass on the surface. The thickness and refractive index of the film determines the particular colors and shades that are observed. Generally, wavelengths of light which reflect from the surface in phase with the incoming light will be additive, or undergo constructive interference, and thus be visible. Wavelengths that reflect from the surface out of phase with the incoming light will be attenuated through destructive interference and will not emerge from the films. Preferably, the wafer 221 comprises silicon, the optical coating comprises silicon nitride, and the attachment polymer comprises a hydrophobic polymer. FIGS. 13 through 16 illustrate how the SILAS surface is used to indicate the presence of a phage marker, utilizing a single greatly enlarged antibody/phage/antibody conjugate 231 representing a large number of such structures which are essentially uniformly distributed over the surface of attachment polymer 224. In FIG. 13, a first antibody 228 specific to a phage marker, such as a capsid protein, are attached to the attachment polymer, the surface 225 of which becomes an immobilization zone. A sample solution is contacted to surface 224. If the specified phage biomarker 230 is present, it attaches to the first antibody 228 as shown in FIG. 14. In FIG. 15, a second detector antibody 232 has been contacted to surface 224 and attaches to the biomarker 230, if the biomarker is present. The second antibody 232 is labeled with a reacting agent, such as horseradish peroxidase (HRP) or alkaline phosphatase. Then an enzyme, such as 3,3′,5,5′ tetramethylbenzidine (TMB), is applied 236 to the surface which reacts with the HRP to form a precipitant 238 which forms a thin film layer 240 (FIG. 16) which alters the color of the surface. As shown in FIG. 16, light 242 impinging on the surface 245 will reflect differently, as shown at 244 and 246, depending on whether or not thin film layer 240 is present. Thus, the combination of the second antibody 232 labeled with the reacting agent, the enzyme, and the optical coating 222 for a color moderator 235 which interacts with the bacteriophage or a biological substance associated with a bacteriophage, whereby the presence of the bacteriophage or the biological substance associated with a bacteriophage causes the immobilization zone 225 to change color.

FIGS. 17 and 18 illustrate the difference in reflectance between a SILAS surface without the thin film layer 240 (FIG. 17) that is for a negative result, and with the thin film layer 240 (FIG. 18) that is for a positive result. The reflectance is higher in the orange-red wavelengths for the negative result, which yields a gold color, and higher in the deep blue wavelengths for the positive result, which yields a purple color. SILAS surfaces are available from Thermo Electron Corporation, 81 Wyman Street, Waltham, Mass. 02454-9046. A description of the SILAS process can be found on the Internet on the Thermo Electron Corporation web site, such as at http://wwwwthermo.com, or more particularly at http://www.thermo.com/com/cda/article/general/1,,53,00.html which publications are incorporated herein by reference to the same extent as though fully disclosed herein.

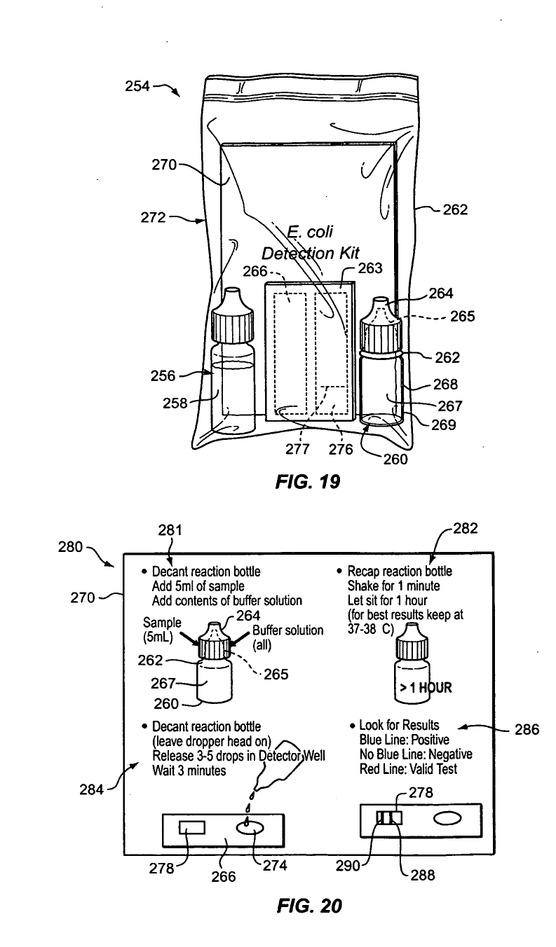

FIG. 19 shows an exemplary test kit 254 for detecting a microscopic living organism, as well as typical directions for using the test kit. See also FIG. 20. Test kit 254 preferably includes a container 256 of buffer solution 258, a reaction container 260, one or more detection elements 266 (FIG. 20) enclosed in a protective case 263, directions 270 for using the kit, and a receptacle 272 for holding the foregoing test kit parts. Protective case 263 may also include a reference detection element 276 indicating the expected result 267 if no bacteria are present. For example, if the detection element is a lateral flow strip, the reference detection element may be an identical lateral flow strip on which a reference sample of bacteriophage has been applied which had no bacteria present. Reaction container 260 includes a container body 267 and a container closure 264. Preferably, the reaction container body is a bottle 267 and the reaction container closure is a bottle cap 264. Reaction container 260 contains phage 268. Phage 268 preferably comprises a predetermined amount of phage that is attached to the interior wall 269 of reaction container body 267. Cap 264 is preferably a screw-on cap having interior threads 262 that mate with threads on the top portion of bottle 267. Cap 264 preferably includes a dispenser 265, which preferably is a dropper head designed to release drops of a predetermined size. In this embodiment, detection element 266 is a lateral flow strip 266, but it also could be a SILAS surface element as described in connection with FIGS. 12-16. Flow strip 266 includes a sample pad 274 (FIG. 20) and a detection window 278. Preferably, receptacle 272 comprises a plastic bag 272, which serves the dual purpose of holding the test kit parts and providing a convenient disposal receptacle after the test is completed.

FIG. 20 shows an exemplary set of directions 270 for using test kit 254. Directions 270 preferably comprise a sheet of paper with printed text and pictures illustrating the test procedure. The directions 270 also illustrate an exemplary method 280 for detecting a microscopic living organism. Method 280 comprises processes 281, 282, 284, and 286. In first process 281, the reaction bottle 267 is decanted by removing cap 264 and dropper head 265 and adding 5 milliliters (ml) of sample. Then the buffer solution 258 is added. In process 282, dropper head 265 and cap 264 are replaced on the bottle 267, the capped reaction container 260 is shaken preferably for a prescribed amount of time, such as one minute, and the solution is then incubated by allowing it to sit for a preferably prescribed amount of time, such as one hour. Cap 264 is then removed and a prescribed amount of the incubated sample is released onto sample pad 278. The user then waits for preferably a predetermined amount of time, such as three minutes. In process 286, the user looks in the detection window 278 for the results. As discussed above, a first line 288 of a first color, such as blue, appears if the sample contains the bacteria for which the test kit is specified. If no line of the first color appears, the test is negative. Optionally, a second line 290, preferably of a second color, appears to indicate that the test is valid. First line 288 corresponds to reagent line 46 of FIG. 3, while second line 290 corresponds to internal control zone 48 of FIG. 3. If a reference detection element 276 (FIG. 19) is used, then the first line 288 may be compared to the reference line 277 to determine if the test is positive or negative. For example, if the first line 288 is clearly a darker blue than the reference line 277, then a positive result is indicated.

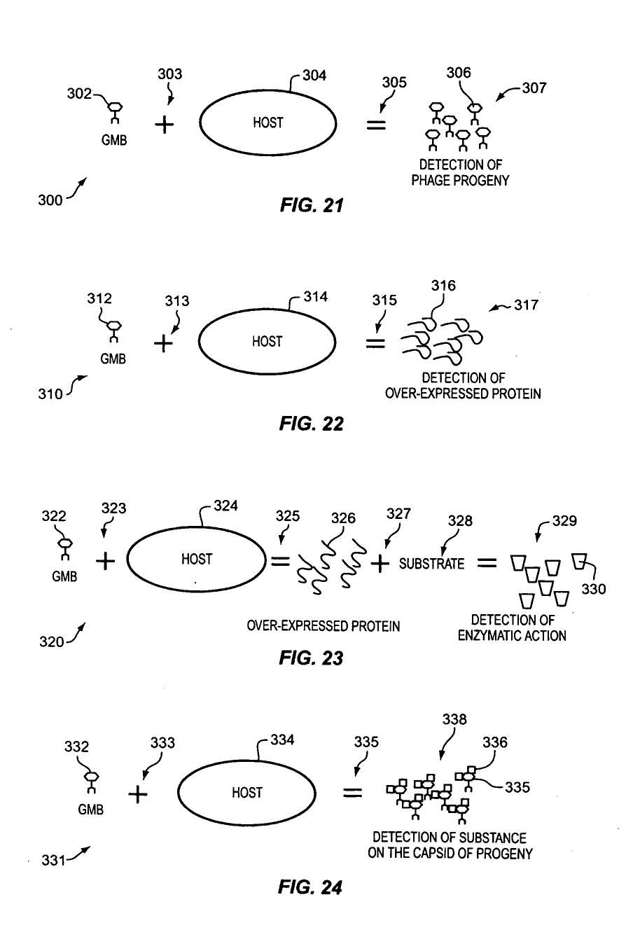

FIG. 21 illustrates an exemplary assay 300 according to the invention utilizing a bacteriophage 302 genetically modified to enhance a desirable property of the infection process. Here, “genetically modified bacteriophage” includes both bacteriophage in which the DNA is modified or manipulated in some manner as well as bacteriophage which has been selectively bred to emphasize certain characteristics. The desirable property can be burst volume, burst time, and infectivity. Burst volume is the quantity of phage that are replicated, burst time is the time it takes the phage to burst the target bacteria, and infectivity is the efficiency of the phage in infecting bacteria, i.e., the percentage of the available bacteria that are infected by a given amount of phage. The phage may be genetically modified to enhance one or more of these desirable properties, and/or other desirable properties. As shown in FIG. 21, the genetically modified phage 302 is used to detect microscopic organisms in the same manner as non-modified phage. That is, an amount of genetically modified phage 302, preferably below the detection limit, is added 303 to a sample of host microorganisms 304, allowed to infect and incubate 305, to generate phage progeny 306, which is detected 307. Because the parent phage 302 is genetically modified to enhance a property of the infection process, the detection can be made faster, more sensitive, and/or more reliable.

FIG. 22 illustrates an exemplary assay 310 according to the invention utilizing a bacteriophage 312 genetically modified to over-express a detectable biomarker, such as a protein. As shown in FIG. 22, the genetically modified phage 312 is used to detect microscopic organisms in the same manner as non-modified phage. That is, an amount of genetically modified phage 312, preferably below the detection limit, is added 313 to a sample of host microorganisms 314, allowed to infect and incubate 315, to generate phage progeny 316, which is detected 317. Because the parent phage 302 is genetically modified to over-express a detectable biomarker, this biomarker is more easily detected, and thus the detection can proceed faster and/or be more sensitive, i.e., detect a lower level of bacteria. Alternatively, this allows a smaller amount of parent phage to be used.

FIG. 23 illustrates an exemplary assay 331 according to the invention utilizing a bacteriophage genetically modified to express an enzyme. As shown in FIG. 23, an amount of genetically modified phage 322, preferably below the detection limit, is added 323 to a sample of host microorganisms 324, allowed to infect and incubate 325, to generate an enzyme 326. The bacteria may or may not lyse. A substrate 328 is added 327, which reacts 329 with the enzyme to produce an enzymatic product 330 or other enzymatic action, which is detected. This genetic modification can also offer alternative detection methods that are more easily performed, can proceed faster and/or be more sensitive, or allow a smaller amount of parent phage to be used.

FIG. 24 illustrates an exemplary assay according to the invention utilizing a bacteriophage genetically modified to express a target on the capsid protein. As shown in FIG. 24, an amount of genetically modified phage 332, preferably below the detection limit, is added 333 to a sample of host microorganisms 334, allowed to infect and incubate 355, to generate progeny 338, which includes a biological marker 336 on the surface of the capsid protein 335. Since the biomarker is on the exterior of the capsid, it may be more easily detected than other markers, and/or can be detected without a phage disassociation process, which can speed up the detection.

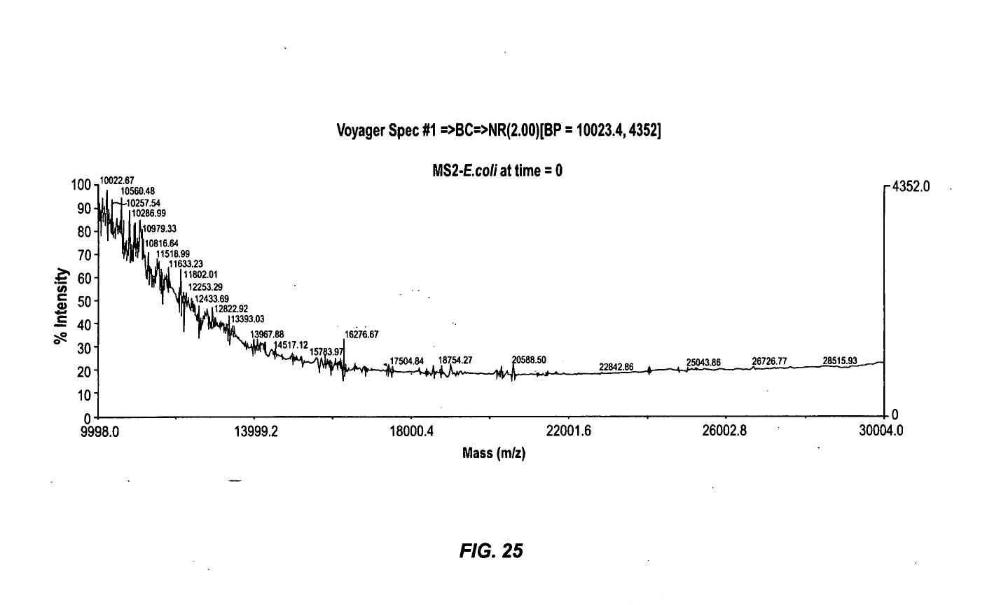

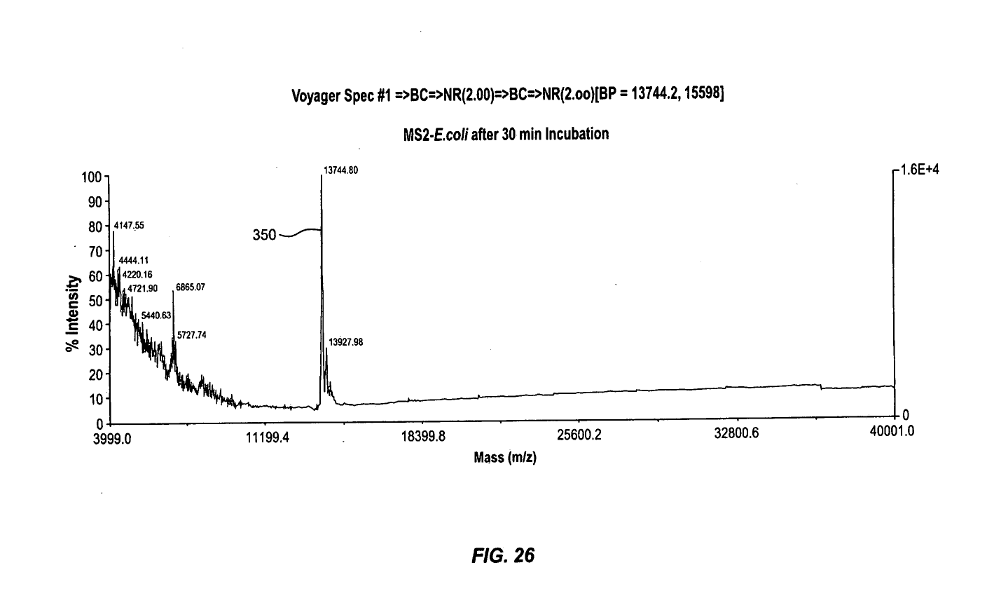

FIG. 25 shows an exemplary MALDI spectrum of an MS2-E. coli bacteriophage infected sample at a time immediately after introduction of the bacteriophage, while FIG. 26 shows an exemplary MALDI spectrum of an MS2-E. coli bacteriophage infected sample at a time 30 minutes after the time of the spectrum of FIG. 25. The effect of bacteriophage amplification is immediately evident in that intensity peak 350 for the bacteriophage marker is greatly amplified. These curves demonstrate the speed and power of bacteriophage amplification.

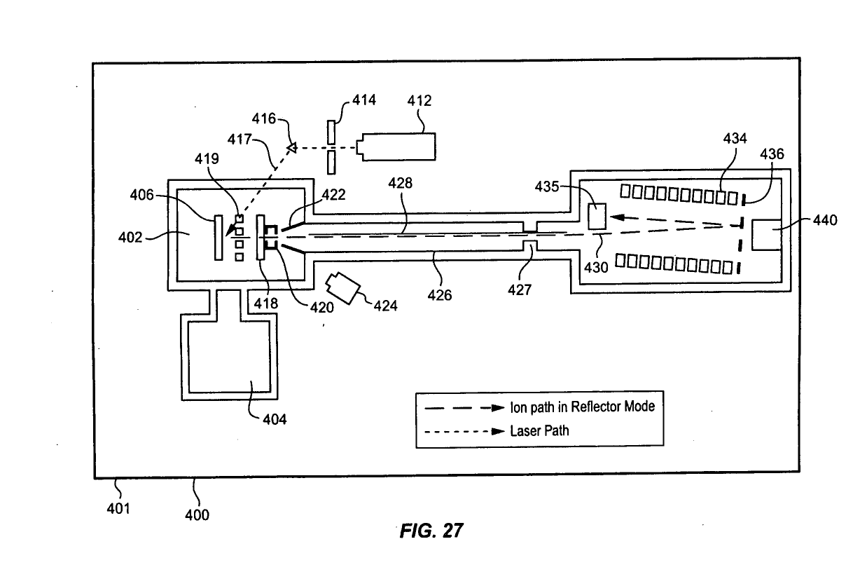

FIG. 27 is a block diagrammatic illustration of a MALDI mass spectrometer 400, which is one of the methods that may be used for performing the detection processes 28, 99, 116, and 130 according to the invention. The spectrometer 400 includes a housing 401, a main source chamber 402, a sample loading chamber 404, a sample plate 406, a laser 412, a laser collimnator or attenuator 414, a prism 416, a ground grid 418, a variable voltage grid 419, an optional collision cell 420, an aperture 422, which is generally grounded, a video camera 424 used for aligning the laser beam on the plate 406, a flight tube 426, a beam guide wire 428, a timed ion selector 427, and a linear detector 440. Optionally, the system 400 can also include a reflector assembly 434, which preferably includes an electrostatic mirror 436, and a reflector detector 435, all of which extends the flight path and the sensitivity of the spectrometer 400. The system 400 operates as follows. A sample plate that has samples 470 (FIG. 28) from the bacteriophage incubation process deposited on it is placed in loading chamber 404 and loaded into the main source chamber. The laser 412, attenuator 414, prism 416, and plate 406 are adjusted so the laser beam 417 will strike the sample. The beam 417 is activated and the sample is exposed to the laser beam. The beam essentially explodes the sample, disintegrating it into its components. Ionized particles from the sample are accelerated by the grids 418 and 419 and collimnated by the aperture 422 and a beam 430 of particles passed down the flight tube 426. The kinetic energy of all particles will be the same, so lighter particles move faster down the flight tube faster and are measured first. The heavier the particle, the longer it takes to arrive at the detector. The detector 440 or 435 measures the particle intensity creating a MALDI intensity spectrum of intensity versus mass, such as shown in FIGS. 11, 25, and 26.

FIG. 28 illustrates an exemplary embodiment of an assay process 440 according to the invention utilizing a MALDI mass spectrometer and immunomagnetic separation. A sample 451 includes biological targets 452 and other biological materials 453. Beads 450 coated with an antibody that is specific to the target 452 are added to the sample 451, which is stirred or otherwise incubated for a predetermined time, such as twenty minutes, to form bead-target complexes 454 in process 455. A magnet 458 is used to isolate the bead-target complexes 454 in process 460. The non-target remainder is decanted 462, and the bead-target complexes are concentrated into a smaller volume of solvent such as water to form concentrated sample 464. The bead-target complexes 454 are then deposited 472 onto a MALDI probe 466, which is generally a plate 466 with raised circular ridges 468 to capture the sample 470. The probe is covered 474 with a matrix solution, then the probe 466 is loaded 478 into the MALDI spectrometer 400 and a mass spectrum 480 is obtained as described above.

### Examples

### A. Lateral Flow Examples

In the following example, the MS2 phage is used to detect E. coli. in the process of FIG. 1. Lateral flow strips, as illustrated in FIGS. 3 and 4, were prepared with polyclonal antibodies that specifically bind to the MS2 phage.

Determining the MS2 Detection Limit of the Lateral Flow Strips—Bacteriophage MS2 (ATCC 15597-B1) was prepared from infected E. coli (ATCC 15597) cells on a confluent plate. The concentration of viable MS2 from this preparation was 2×10⁷ pfu/mL by plaque assay. A dilution series of this MS2 stock was made to produce a range of from 1×10⁷ pfu/mL to 1×10⁵ pfu/mL. MS2 was detected with the lateral flow strips. Results are shown in Table 2.

TABLE 2

| | MS2 Concentration | Lateral Flow Result |
|---|---|---|
| MS2 Dilution | (pfu/mL) | (line intensity) |
| 1/2 | 1 × 10⁷ | + |
| 1/20 | 1 × 10⁶ | +/− |
| 1/200 | 1 × 10⁵ | − |

Line intensity was determined visually 15 minutes after loading a sample into a lateral flow strip. Line intensity was ranked on a scale ranging from “++” indicating maximum line intensity to “−” indicating no detectable line. “+/−” indicates a line that is barely detectable. The results of this assay indicate that the detection limit of MS2 on the lateral flow strips prepared for these tests is 1×10⁶ pfu/mL.

Determining E. coli Detection Limits and Total Testing Times—E. coli (ATCC 15597) from a saturated culture were diluted to produce raw samples having concentrations of 1×10⁸, 1×10⁷, 1×10⁶, 1×10⁵, 1×10⁴, 1×10³, and 1×10² cells/mL. MS2 was added to each raw sample to give test samples with MS2 concentrations of 1×10⁶ pfu/mL. The test samples were incubated for 1 hour to 5 hours at 37° C. After incubation, the test samples were diluted 10:1 such that the parent MS2 concentration was 1×10⁵ pfu/mL—below the established detection limit. The test samples were then analyzed using the lateral flow strips. The results are shown in Table 3.

TABLE 3

| E. coli | E. coli in | Lateral Flow Result | | | |
|---|---|---|---|---|---|
| Concentration | Reaction | (line intensity) | | | |
| (cells/mL) | (cells) | 1 hour | 2 hours | 3 hours | 4 hours |
| 1 × 10⁷ | 1 × 10⁶ | + | ++ | ++ | ++ |
| 1 × 10⁶ | 1 × 10⁵ | + | ++ | ++ | ++ |
| 1 × 10⁵ | 1 × 10⁴ | − | + | ++ | ++ |
| 1 × 10⁴ | 1 × 10³ | − | − | + | ++ |
| 1 × 10³ | 1 × 10² | − | − | − | + |

These results show that 100 E. coli cells can be detected after 4 hours using the disclosed invention. Higher levels of E. coli (1×10⁵ cells or more) are detectable after 1 hour.

Similarly, the lateral flow strip process described above has also been used successfully to detect the salmonella choleraesuis bacteria using the PRD-1 bacteriophage, the bacillus anthracis bacteria using the gamma phage, and the group B Streptococcus using the B-30 bacteriophage.

### B. MALDI Examples

Bacteria was detected in mixtures by bacteriophage amplification coupled with MALDI. This example demonstrates the detection of Bacillus anthracis, Escherichia coli, and group B Streptococcus (streptococcus agalactiae or B. agalactiae) ensconced in bacterial mixtures using BA-MALDI-MS and the bacteriophages gamma phage, MS-2, B-30, respectively. The stern strain of Bacillus anthracis, Escherichia coli, gamma phage, and coliphage MS2 were provided by the Armed Forces Institute of Pathology (Washington, D.C.). Streptococcus agalactiae and phage B30 were kindly provided by Dr. David G. Pritchard at the University of Alabama, Birmingham. B. anthracis and E. coli were cultured in BHI broth, while B. agalactiae was grown in Todd-Hewitt broth. Phage amplification experiments were conducted according to the above protocols. Briefly, phage was added to suspensions containing targeted bacteria at concentrations below the detection limit of the respective phage. After incubation for an appropriate amount of time, aliquots of the various suspensions were subjected to mass analysis to determine if phage amplification had occurred.

MALDI spectra of the three phages used in this study showed characteristic, reproducible peaks that allowed each phage to be identified by a unique protein mass spectral profile. The mass spectrum of the gamma phage showed peaks at 22.87 kDa and 31.90 kDa, the mass spectrum of coliphage MS2 showed a peak at 13.75 kDa, and phage B30 showed mass spectral peaks at 13.32 kDa and 8.07 kDa. The data showed that when suspensions were generated with multiple species of bacteria, the respective phages were able to type their hosts as determined by the appearance of phage mass spectral peaks where none were present upon initial infection. When multiple phages were added to a suspension containing a single species of bacteria, the proper phage was able to infect its host and generate progeny to the point that the phage became detectable by MALDI. Furthermore, when the phages were added to suspensions containing multiple species of bacteria, the presence of each bacterium could be ascertained simultaneously by visualizing the appearance of the respective phage protein profiles on the MALDI spectrum.

Similarly, detection of a CDC vaccine strain of yersina pestis (Y. pestis) was detected using bacteriophage A1122, both provided by the Armed Forces Institute of Pathology (Washington, D.C.) and a MALDI process as described above. Other examples that have been successfully performed include detection of staphylococcus aureus using bacteriophage 47, and separately using bacteriophage 187, and again separately using bacteriophage 53. As another example, salmonella choleraesuis was detected using PRD-1 bacteriophage and the MALDI process. This bacteria was also detected using the P-22 bacteriophage and MALDI. In addition, Escherichia coli was also detected using the PRD-1 bacteriophage, the T-4 bacteriophage, and the T-1 bacteriophage, separately, using MALDI.

In another set of experiments, it was shown that use of bacteriophage reduces the detection limit for E. coli and that the technique of adding an amount of bacteriophage below the detection limit is effective. In this embodiment, a bacteria target-bead complex was used to isolate E. coli from solution, as described in United States Patent Application Publication US 2002/0192676 A1, which is incorporated herein by reference to the same extent as though fully disclosed herein. The bacteria target-bead complex was re-suspended in a solution containing the MS2 bacteriophage, with the bacteriophage concentration adjusted so that the ion signal from the capsid protein of the MS2 bacteriophage was below the detection limit of the MALDI mass spectrometer. After a forty-minute incubation period, an aliquot of the solution was removed and analyzed by an on-probe MALDI procedure for the 1 3 kDa capsid protein. The [M+H]⁺ (m/z 13,726) and [M+2H]⁺² (m/z 6865) ion signals for the MS2 capsid protein were easily detected. When this process was performed for a concentration of 5.0×10⁶ E. coli cells per ml., the mass spectrum included protein signals for both the E. coli bacterium and the MS2 bacteriophage. The process was repeated for decreasing concentrations of E. coli. For concentrations of E. coli of 5.0×10⁵ E. coli cells per ml and concentrations of 5.0×10⁴ E. coli cells per ml, the mass spectrum failed to show any definite protein signals for the E. coli cells but clearly showed protein signals for the MS2 bacteriophage capsid protein. Target bacteria concentrations as low as 1.0×10³ cells per ml have been detected by this process.

### C. Antibiotic Resistance

In this example, the minimum inhibitory concentration (MIC) of antibiotics in Staphylococcus aureus (S. aureus) was rapidly determined by bacteriophage amplification with MALDI-MS. The MIC is the lowest concentration of antibiotic that inhibits the growth of a particular strain of S. aureus. If the strain is sensitive to the antibiotic at the assayed concentration, no bacteriophage biomarker signal will be detected by MALDI-MS due to antibiotic inhibition, subsequently suppressing phage amplification. Conversely, if the phage biomarker signal is detected, the MIC has not been attained and represents the point where the antibiotic is ineffective. Streptomycin and tetracycline were selected to determine MIC for this study.

Twenty-four hour cultures of S. aureus (ATCC 27709, Manassas Va.) were grown in BHI broth with and without antibiotics (streptomycin and tetracycline). Phage 187 and S. aureus samples were diluted below the detection limit of MALDI followed by infection of the phage into the host, where amplification of the bacteriophage occurred until the cells burst. The samples were then cleaned and concentrated for analysis. Using the dried droplet method, the sample was plated for analysis onto a hydrophobic target plate with a 15 mg/mL ferulic acid matrix mixed in a solution of formic acid, acetonitrile, and HPLC grade water. Mass spectra were obtained in linear mode using a MALDI-TOF-MS PerSeptive Biosystems Voyager-DE STR Biospectrometry Workstation (Framingham, Mass.).

By growing the strain of S. aureus in the antibiotics, either the concentration of antibiotic will be sufficient to destroy the bacteria cells or not. If the bacterial cells are viable, meaning that they are unaffected by the antibiotic, then phage amplification occurs when the cells are infected. The result is that the protein marker for the bacteriophage is seen in the mass spectrum. On the other hand, if the biomarker is not seen in the mass spectrum, then this indicates that the minimum inhibitory concentration has been reached or exceeded. The conclusion is that the cells have been destroyed, and therefore no bacteriophage amplification can occur.

Semi-purified techniques were used to filter and concentrate the samples before analysis on MALDI. Due to the intolerance of salts by the instrument, the samples were spin filtered using a 100 kDa cutoff. The mass spectrum from phage 187 showed a distinctive protein biomarker at 15,245 Da. Phage 187 was also purified by ultracentrifugation and a cesium chloride gradient, which confirms the biomarker identified by semi-purified filtering techniques. The MALDI MS limit of detection was established for both the bacteria and phage to be 10⁶ cells/mL and 10⁸ phage/mL, respectively. To verify that the signal was from the amplified phage, the concentrations of bacteria and phage were kept below the MALDI limit of detection during the experiment. At low concentrations of the antibiotics, the protein peak from the phage was present in the MALDI spectrum, indicating that bacterial growth and phage replication was still occurring. At higher concentrations, the spectrum was void of the protein peak, indicating that the MIC had been met or exceeded.

### D. SILAS Surface Example

Methods: Coated surfaces and HRP conjugated antibody were prepared using standard methods developed at Thermo Electron Corporation, 81 Wyman Street, Waltham, Mass. 02454-9046. Briefly, the surfaces were coated in a solution of HEPES buffer at pH 7.8 containing 4 ug/ml Rabbit anti-MS2 antibody for 48 hours. After coating, the wafers were washed and over-coated with a sugar:protein preservative, then divided into chips 7 mm square.

Conjugation proceeded according to a Thermo Electron modification of the method of Nakane. HRP was activated using sodium periodate to introduce aldehydes onto the carbohydrate portion of the protein. The activated HRP and the rabbit anti-MS2 antibody were mixed and allowed to incubate. The conjugates were stabilized by adding sodium borohydride to reduce the Schiff's bases.

Testing was performed to determine the ability to detect the MS2 provided. Initial formats included both simultaneous and sequential formats. The simultaneous format consisted of mixing sample and conjugate (diluted 1:100 in conjugate diluent) and adding the sample to the surface of the coated OIA chip. Following incubation, the surface was washed and dried followed by addition of enzyme substrate (TMB). First and second incubations were kept equivalent at either 5 or 10 minutes. The sequential assay was similar to the simultaneous assay, except the sample and conjugate were not mixed but added to the chip independently. Incubation steps were separated by washing and blotting steps. The sequential assay was run using 10-minute incubations for all steps.

Results: The un-optimized methods described here were clearly able to detect the MS2 sample at 10⁷ but the results were mixed for 10⁴. The color change in all cases was from a deep gold-orange to a dark purple. Using the simultaneous format, weak, barely visible results were detected in undiluted sample using two ten-minute incubations. Using the sequential method, the signal detected was stronger at 10⁷. With three ten-minute incubations, the 10⁷ signal was clear and the 10⁴ signal was visible, but very weak. A dilution series was performed by diluting the 10⁷ sample in saline using 2-fold dilutions. Positive results were detected out to a 1:16 dilution of the 10⁷ sample. This would equate to roughly 6.25×10⁵.

It is a feature of the preferred embodiment of the invention that the parent phage are not destroyed, removed, neutralized, or inactivated in the bacteriophage exposed sample. In prior art methods, the extracellular bacteriophage, that is the bacteriophage outside the bacteria or other microorganism being infected, are at some point destroyed, removed, neutralized, or inactivated. This is not required in the present invention. In particular, the destruction, neutralization, or inactivation of the extracellular bacteriophage by the addition of an agent that kills the bacteriophage is preferably not done, as this unnecessarily complicates the method and can affect the progeny bacteriophage if the agent is not removed or neutralized.

There has been described a microorganism detection method which is specific to a selected organism, sensitive, simple, fast, and/or economical, and having numerous novel features. The invention can be used in a wide variety of applications including human clinical diagnostics, veterinary diagnostics, food pathogen detection, environmental testing, and biowarfare detection. It should be understood that the particular embodiments shown in the drawings and described within this specification are for purposes of example and should not be construed to limit the invention, which will be described in the claims below. Further, it is evident that those skilled in the art may now make numerous uses and modifications of the specific embodiment described, without departing from the inventive concepts. Equivalent structures and processes may be substituted for the various structures and processes described; the subprocesses of the inventive method may, in some instances, be performed in a different order; or a variety of different materials and elements may be used. Consequently, the invention is to be construed as embracing each and every novel feature and novel combination of features present in and/or possessed by microorganism detection apparatus and methods described.

What is claimed as new and desired to be protected by Letters Patent of the United States is:

1-52. (canceled)

53. A method of detecting the presence or absence of a target microorganism in a sample to be tested, said method comprising:
(a) combining with said sample, bacteriophage capable of infecting said target microorganism to create a bacteriophage exposed sample;
(b) providing conditions to said bacteriophage exposed sample sufficient to allow said bacteriophage to infect said target microorganism and to multiply in said target to create a detectable amount of a capsid protein associated with said bacteriophage in said bacteriophage exposed sample; and
(c) assaying said bacteriophage exposed sample to determine the presence or absence of said capsid protein associated with said bacteriophage as an indication of the presence or absence of said target microorganism.

54. A method as in claim 53 wherein said microorganism is a bacterium and said assaying comprises detecting said capsid protein as an indication of the presence of said target bacterium in said sample.

55. A method as in claim 53 wherein said providing comprises actively lysing said microorganism prior to said assaying.

56. A method as in claim 55 wherein said actively lysing comprises a method selected from the group consisting of: adding chloroform to said bacteriophage exposed sample; treating said bacteriophage exposed sample with acid; and physically processing said bacteriophage exposed sample.

57. A method as in claim 53 wherein said providing further comprises dissociating said bacteriophage.

58. A method as in claim 57 wherein said dissociating comprises adding a dissociating agent to said bacteriophage exposed sample.

59. A method as in claim 53 wherein said combining comprises tagging said parent bacteriophage.

60. A method as in claim 59 wherein said assaying comprises removing said tagged parent bacteriophage from said bacteriophage exposed sample.

61. A method as in claim 53 wherein said combining includes tagging the capsid protein of said parent bacteriophage.

62. A method as in claim 53 wherein said assaying comprises providing a reference indicating an assay result if said target microorganism are not present in said sample and comparing a corresponding result from said bacteriophage exposed sample to said reference result.

63. A method of detecting the presence or absence of target microorganism in a sample to be tested, said method comprising:
(a) combining with said sample, parent bacteriophage capable of infecting said target microorganism to create a bacteriophage exposed sample;
(b) providing conditions to said bacteriophage exposed sample sufficient to: allow said bacteriophage to infect said target microorganism and multiply in said target microorganism to create progeny bacteriophage; and produce a dissociated bacteriophage substance accessible to an assay; and
(c) assaying said bacteriophage exposed sample to determine the presence or absence of said bacteriophage substance as an indication of the presence or absence of said target microorganism in said sample.

64. A method as in claim 63 wherein said microorganism is a bacterium and said assaying comprises detecting said bacteriophage substance as an indication of the presence of said target bacterium in said sample.

65. A method as in claim 63 wherein said bacteriophage substance is a capsid protein.

66. A method as in claim 63 wherein said providing comprises lysing said microorganism to release said bacteriophage.

67. A method as in claim 66 wherein said lysing comprises adding a microbial lysozyme to said bacteriophage exposed sample.

68. A method as in claim 66 where said lysing comprises a method selected from the group consisting of: adding chloroform to said bacteriophage exposed sample; treating said bacteriophage exposed sample with acid; and physically processing said bacteriophage exposed sample.

69. A method as in claim 63 wherein said dissociating comprises adding a dissociating agent to said bacteriophage exposed sample.

70. A method as in claim 69 wherein said adding comprises adding a substance selected from the group consisting of: acid, urea, denaturing agents, and enzymes.

71. A method as in claim 63 wherein said combining comprises tagging said parent bacteriophage and said providing includes segregating said tagged parent bacteriophage and then dissociating said bacteriophage to produce said dissociated bacteriophage substance.

72. A method as in claim 71 wherein said tagging comprises a process selected from the group consisting of: biotinylating said parent bacteriophage; and attaching said parent bacteriophage to a physical substrate.

73-114. (canceled)
